(19) United States
(12) Patent Application Publication
(10) Pub. No.: US 2008/0261977 A1
(43) Pub. Date: Oct. 23, 2008

(54) Pyrimidine Derivatives as Cannabinoid Receptor Modulators

(76) Inventors: Andrew John Eatherton, Essex (GB); Gerard Martin Paul Giblin, Essex (GB); William Leonard Mitchell, Essex (GB); Alan Naylor, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 10/597,521

(22) PCT Filed: Feb. 21, 2005
(86) PCT No.: PCT/EP2005/001939

§ 371 Date: Jul. 28, 2006

(30) Foreign Application Priority Data
Feb. 23, 2004 (GB) 0403998.8
Nov. 12, 2004 (GB) 0425071.8

(51) Int. Cl. A61K 31/5377; C07D 239/42; A61K 31/505; A61P 29/00; A61P 37/00; C07D 413/06

(52) U.S. Cl. 514/235.8; 544/331; 514/275; 544/122

(57) ABSTRACT

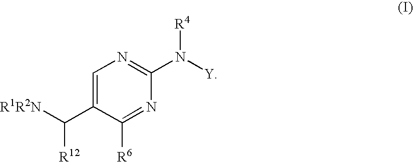

The present invention relates to novel pyrimidine derivatives such as compounds of the formula (I) and the use of such compounds or pharmaceutical compositions thereof in the treatment of diseases, particularly pain, which are mediated by the activity of the cannabinoid 2 receptor.

The present invention relates to novel pyrimidine derivatives, pharmaceutical compositions containing these compounds and their use in the treatment of diseases, particularly pain, which diseases are caused directly or indirectly by an increase or decrease in activity of the cannabinoid receptor.

Cannabinoids are a specific class of psychoactive compounds present in Indian cannabis (Cannabis sativa), including about sixty different molecules, the most representative being cannabinol, cannabidiol and several isomers of tetrahydrocannabinol. Knowledge of the therapeutic activity of cannabis dates back to the ancient dynasties of China, where, 5,000 years ago, cannabis was used for the treatment of asthma, migraine and some gynaecological disorders. These uses later became so established that, around 1850, cannabis extracts were included in the US Pharmacopaeia and remained there until 1947.

Cannabinoids are known to cause different effects on various systems and/or organs, the most important being on the central nervous system and on the cardiovascular system. These effects include alterations in memory and cognition, euphoria, and sedation. Cannabinoids also increase heart rate and vary systemic arterial pressure. Peripheral effects related to bronchial constriction, immunomodulation, and inflammation have also been observed. The capability of cannabinoids to reduce intraocular pressure and to affect respiratory and endocrine systems is also well documented. See e.g. L. E. Hollister, Health Aspects of Cannabis, Pharmacological Reviews, Vol. 38, pp. 1-20, (1986). More recently, it was found that cannabinoids suppress the cellular and humoral immune responses and exhibit antiinflammatory properties. Wirth et al., Antiinflammatory Properties of Cannabichrome, Life Science, Vol. 26, pp. 1991-1995, (1980).

In spite of the foregoing benefits, the therapeutic use of cannabis is controversial, both due to its relevant psychoactive effects (causing dependence and addiction), and due to manifold side effects that have not yet been completely clarified. Although work in this field has been ongoing since the 1940's, evidence indicating that the peripheral effects of cannabinoids are directly mediated, and not secondary to a CNS effect, has been limited by the lack of receptor characterization, the lack of information concerning an endogenous cannabinoid ligand and, until recently, the lack of receptor subtype selective compounds.

The first cannabinoid receptor was found to be mainly located in the brain, in neural cell lines, and, only to a lesser extent, at the peripheral level. In view of its location, it was called the central receptor (“CB1”). See Matsuda et al., “Structure of a Cannabinoid Receptor and Functional Expression of the Cloned cDNA,” Nature Vol. 346, pp. 561-564 (1990. The second cannabinoid receptor (“CB2”) was identified in the spleen, and was assumed to modulate the non psychoactive effects of the cannabinoids. See Munro et el., “Molecular Characterization of a Peripheral Receptor for Cannabinoids,” Nature, Vol. 365, pp. 61-65 (1993).

Recently, some compounds have been prepared which are capable of acting as agonists on both the cannabinoid receptors. For example, use of derivatives of dihydroxypyrrole-(1,2,3-d,e)-1,4-benzoxazine in the treatment of glaucoma and the use of derivatives of 1,5-diphenyl-pyrazole as immunomodulators or psychotropic agents in the treatment of various neuropathologies, migraine, epilepsy, glaucoma, etc are known. See U.S. Pat. No. 5,112,820 and EP 576357, respectively.

However, because these compounds are active on both the CB1 and CB2 receptor, they can lead to serious psychoactive effects.

The foregoing indications and the preferential localization of the CB2 receptor in the immune system confirms a specific role of CB2 in modulating the immune and antiinflammatory response to stimuli of different sources.

The total size of the patient population suffering from pain is vast (almost 300 million), dominated by those suffering from back pain, osteo-arthritic pain and post-operative pain. Neuropathic pain (associated with neuronal lesions such as those induced by diabetes, HIV, herpes infection, or stroke) occurs with lower, but still substantial prevalence, as does cancer pain.

The pathogenic mechanisms that give rise to pain symptoms can be grouped into two main categories:
those that are components of inflammatory tissue responses (Inflammatory Pain);
those that result from a neuronal lesion of some form (Neuropathic Pain).

Chronic inflammatory pain consists predominantly of osteoarthritis, chronic low back pain and rheumatoid arthritis. The pain results from acute and on-going injury and/or inflammation. There may be both spontaneous and provoked pain.

There is an underlying pathological hypersensitivity as a result of physiological hyperexcitability and the release of inflammatory mediators which further potentiate this hyperexcitability. CB2 receptors are expressed on inflammatory cells (T cells, B cells, macrophages, mast cells) and mediate immune suppression through inhibition of cellular interaction/inflammatory mediator release. CB2 receptors may also be expressed on sensory nerve terminals and therefore directly inhibit hyperalgesia.

The role of CB2 in immunomodulation, inflammation, osteoporosis, cardiovascular, renal and other disease conditions is now being examined. In light of the fact that cannabinoids act on receptors capable of modulating different functional effects, and in view of the low homology between CB2 and CB1, the importance of developing a class of drugs selective for the specific receptor sub-type is evident. The natural or synthetic cannabinoids currently available do not fulfil this function because they are active on both receptors.

Based on the foregoing, there is a need for compounds which are capable of selectively modulating the receptor for cannabinoids and, therefore, the pathologies associated with such receptors. Thus, CB2 modulators offer a unique approach toward the pharmacotherapy of immune disorders, inflammation, osteoporosis, renal ischemia and other pathophysiological conditions.

The present invention provides novel pyrimidine derivatives of formula (I) and pharmaceutically acceptable derivatives thereof, pharmaceutical compositions containing these compounds or derivatives, and their use as CB2 receptor modulators, which are useful in the treatment of a variety of disorders.

The present invention further comprises a method for treating disease mediated by CB2 receptors in an animal, including humans, which comprises administering to an animal in need thereof an effective amount of a compound of formula (I) or a pharmaceutically acceptable derivative thereof.

The invention provides compounds of formula (I):

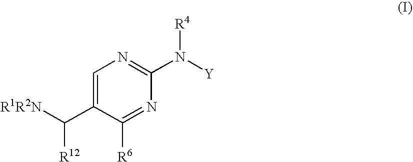

wherein:

Y is phenyl, unsubstituted or substituted with one, two or three substituents;

R¹ is selected from hydrogen, C₁₋₆ alkyl, C₃ cycloalkyl, or halosubstitutedC₁— alkyl;

R² is (CH₂)ₘR³ where m is 0 or 1;

or R¹ and R² together with N to which they are attached form an unsubstituted or substituted 4- to 8-membered non-aromatic heterocyclyl ring;

R³ is hydrogen, an unsubstituted or substituted 4- to 8-membered non-aromatic heterocyclyl group, an unsubstituted or substituted C₃₋₈ cycloalkyl group, an unsubstituted or substituted straight or branched C₁₋₁₀ alkyl, an unsubstituted or substituted C₅₋₇ cycloalkenyl, R¹; or R³ is an unsubstituted or substituted 5- to 6-membered aromatic heterocyclyl group, or group A:

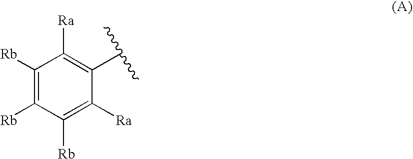

R⁴ is selected from hydrogen, C₁₋₆ alkyl, C₃₋₄ cycloalkyl, or halosubstitutedC₁₋₆ alkyl, COCH₃, or SO₂Me;

R⁵ is

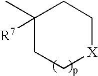

wherein p is 0, 1 or 2, and X is CH₂, O, S, SO or SO₂;

R⁶ is halo, an substituted or unsubstituted (C₁₋₆)alkyl, (C₃₋₆)cycloalkyl, 4- to 7-membered non aromatic heterocyclyl group;

R⁷ is OH, C₁₋₆alkoxy, NR^{8a}R^{8b}, NHCOR⁹, NHS % R⁹, SOqR⁹;

R^{8a} is H or C₁₋₆alkyl;

R^{8b} is H or C₁₋₆alkyl;

R⁹ is C₁₋₆alkyl;

Ra is independently selected from hydrogen, fluoro, chloro or trifluoromethyl;

Rb is independently selected from hydrogen, C₁₋₆alkyl, C₁₋₆ alkoxy, haloC₁₋₆ alkoxy, hydroxy, cyano, halo, sulfonyl, CONH₂, COOH or NHCOOC₆₋₄alkyl;

R¹² is hydrogen or C₁₋₆alkyl;

q is 0, 1 or 2;

and pharmaceutically acceptable derivatives thereof,

wherein the compound is not (5-{[bis-(2-methoxy-ethyl)-amino]-methyl}-4-trifluoromethyl-pyrimidin-2-yl)-3-chlorophenyl)amine or {1-[2-(3-chloro-phenylamino)-4-trifluoromethyl-pyrimidin-5-ylmethyl]-piperidin-4-yl}-methanol, formate.

(5-{[Bis-(2-methoxy-ethyl)-amino]-methyl}-4-trifluoromethyl-pyrimidin-2-yl)-(3-chlorophenyl)-amine and {1-[2-(3-chloro-phenylamino)-4-trifluoromethyl-pyrimidin-5-ylmethyl]-piperidin-4-yl}-methanol, formate do not appear to have any potency or efficacy against CB2.

In one embodiment Y is a substituted phenyl. In one embodiment Y is substituted by 1 or 2 substituents.

When Y is substituted, the substituent or substituents may be selected from: C₁₋₆ alkyl, halosubstitutedC₆₋₄ alkyl, C₁₋₆ alkoxy, a hydroxy group, a cyano group, halo, a C₁₋₆alkyl sulfonyl group, —CONH₂, —NHCOC₁₋₆alkyl, —CH₂COOH, —COOH, halosubstituted C₁— alkoxy, SC₁₋₆alkyl or SO₂NR^{8a}R^{8b} wherein R^{8a} and R^{8b} are as defined above.

In one embodiment Y is substituted by halo, cyano, methyl, trifluoromethyl, methoxy or trifluoromethoxy or SCH₃. In one embodiment halo is chloro, fluoro, or bromo.

In one embodiment the compounds of formula (I) are compounds of formula (Ia):

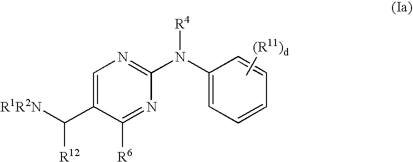

wherein;

R¹ is selected from hydrogen, C₁₋₆ alkyl, C₃₋₆ cycloalkyl and halosubstitutedC₁₋₆ alkyl;

R² is (CH₂)ₘR³ where m is 0 or 1;

or R¹ and R² together with N to which they are attached form a 4- to 8-membered non-aromatic ring selected from azetidinyl, pyrrolidinyl, morpholinyl, piperizinyl, piperidinyl, thiomorpholinyl, tetrahydropyridinyl, azapine, oxapine, azacyclooctanyl, azaoxacyclooctanyl and azathiacyclooctanyl any of which can be unsubstituted or substituted by one, two or three substituents selected from C₁₋₆ alkyl, C₁₋₆ alkylOH, C₁₋₆ alkoxy, a hydroxy group, a cyano group, halo, sulfonyl group, methylsulfonyl, NR^{8a}R^{8b}, NHCOCH₃, (═O), —CONHCH₃ and NHSO₂CH₃, C(O)OC₁₋₆alkyl.

R³ is hydrogen, 2- or 3-azetidinyl, oxetanyl, thioxetanyl, thioxetanyl-s-oxide, thioxetanyl-s,s-dioxide, dioxalanyl, pyrrolidinyl, tetrahydrofuranyl, tetrahydrothiophenyl, tetrahydrothiophenyl-s-oxide, tetrahydrothiophenyl-s,s-dioxide, morpholinyl, piperidinyl, piperazinyl, tetrahydropyranyl, tetrahydrothiopyranyl, tetrahydrothiopyranyl-s-dioxide, tetrahydrothiopyranyl-s,s-dioxide, thiomorpholinyl, thiomorpholinyl-s,s-dioxide, tetrahydropyridinyl, dioxanyl, tetrahydrothiopyran 1,1 dioxide, azapine, oxapine, azacyclooctanyl, azaoxacyclooctanyl, azathiacyclooctanyl, oxacylcooctanyl, thiacyclooctanyl, a C₃₋₈ cycloalkyl group, a straight or branched C₁₋₁₀ alkyl, a C₅₋₇ cycloalkenyl or R⁵, any of which can be unsubstituted or substituted by one, two or three substituents selected from C₁₋₆ alkyl, C₁₋₆ alkoxy, a hydroxy group, a cyano group, halo, sulfonyl group, methylsulfonyl, NR^{8a}R^{8b}, NHCOCH₃, (═O), and —CONHCH₃ and when R³ is alkyl it can be phenyl or phenyl substituted by halo, hydroxy or cyano;

or R³ is group A or selected from furanyl, dioxalanyl, pyrrolyl, oxazolyl, thiazolyl, imidazolyl, oxadiazolyl, thiadiazolyl, triazolyl, triazinyl, isothiazolyl, isoxazolyl, thienyl, pyrazolyl, tetrazolyl, pyridinyl, pyrizinyl, pyrimidinyl, pyrazinyl, triazinyl, or tetrazinyl any of which can be unsubstituted or substituted by one, two or three substituents selected from C₁₋₆ alkyl, C₁₋₆ alkoxy, a hydroxy group, a cyano group, halo, sulfonyl group, methylsulfonyl, NR^{8a}R^{8b}, NHCOCH₃, (═O), and —CONHCH₃;

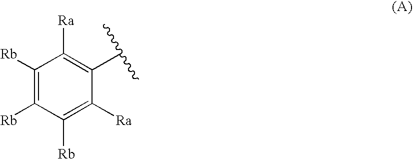

R¹¹ is selected from C₁₋₆ alkyl, halosubstitutedC₁₋₆ alkyl, C₁₋₆ alkoxy, a hydroxy group, a cyano group, halo, a C₁₋₆alkyl sulfonyl group, —CONH₂, —NHCOC₁₋₆alkyl —COOH, —CH₂COOH, halosubstitutedC₁₋₆ alkoxy, SC₁₋₆alkyl and SO₂NR^{8a}R^{8b};

R⁴ is selected from hydrogen, C₁₋₆ alkyl, C₃₋₆ cycloalkyl, or halosubstitutedC₁₋₆ alkyl, COCH₃, and SO₂Me;

R⁵ is

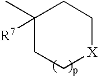

wherein p is 0, 1 or 2 and X is CH₂, O, S, SO or SO₂;

R⁶ is halo, a substituted or unsubstituted (C₁₋₆)alkyl, (C₃₋₆)cycloalkyl, 4 to 7-membered non aromatic heterocyclyl group;

R⁷ is OH, C₁₋₆alkoxy, NR^{8a}R^{8b}, NHCOR⁹, NHSO₂R⁹, SOqR⁹;

R^{8a} is H or C₁₋₆alkyl;

R^{8b} is H or C₁₋₆alkyl;

R⁹ is C₁₋₆alkyl;

R¹² is hydrogen or C₁₋₆alkyl;

Ra is independently selected from hydrogen, fluoro, chloro or trifluoromethyl;

Rb is independently selected from hydrogen, C₁₋₆ alkyl, C₁₋₆ alkoxy, haloC₁₋₆ alkoxy, hydroxy, cyano, halo, sulfonyl, CONH₂, COOH or NHCOOC₁₋₆alkyl;

q is 0, 1 or 2;

d is 0, 1, 2 or 3

and pharmaceutically acceptable derivatives thereof

wherein the compound is not

(5-{[bis-(2-methoxy-ethyl)-amino]-methyl}-4-trifluoromethyl-pyrimidin-2-yl)-(3-chlorophenyl)-amine or {1-[2-(3-chloro-phenylamino)-4-trifluoromethyl-pyrimidin-5-ylmethyl]-piperidin-4-yl}-methanol, formate.

In one embodiment R¹ is hydrogen or methyl.

In one embodiment R⁴ is C₁₋₆alkyl or hydrogen, suitably methyl or hydrogen, even more suitably hydrogen.

In one embodiment R⁶ is C₁₋₆alkyl, (C₃₋₆)cycloalkyl or CF₃.

In one embodiment R⁷ is OH.

In one embodiment X is CH₂.

In one embodiment, R³ is an unsubstituted or substituted 4- to 8-membered non-aromatic heterocyclyl group, an unsubstituted or substituted C₃₋₈ cycloalkyl group, an unsubstituted or substituted straight or branched C₁₋₁₀ alkyl, an unsubstituted or substituted C₅₋₇ cycloalkenyl, R⁵; or R³ is an optionally substituted 5- to 6-membered aromatic heterocyclyl group, or group A.

In one embodiment when R³ is an optionally substituted C₃₋₈cycloalkyl group or an optionally substituted 4- to 8-membered nonaromatic heterocyclyl group, an unsubstituted or substituted 5- to 6-membered aromatic heterocyclyl group, or group A, m is 1.

In one embodiment R² is CH₂R₃.

In one embodiment R¹² is hydrogen.

In one embodiment R³ is an unsubstituted or substituted 4- to 8-membered non-aromatic heterocyclyl group or group A, pyridinyl, or pyrimidinyl, any of which can be optionally substituted.

In one embodiment R¹ and R² together with N to which they are attached form a 4- to 8-membered non-aromatic ring selected from azetidinyl, pyrrolidinyl, morpholinyl, piperizinyl, piperidinyl, tetrahydropyridinyl, azapine, oxapine, azacyclooctanyl, azaoxacyclooctanyl and azathiacyclooctanyl.

In one embodiment compounds of formula (I) are compounds of formula (Ib):

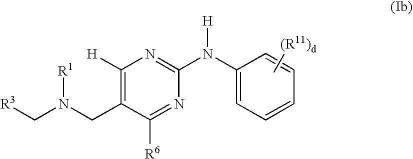

R¹ is hydrogen or methyl

R³ is an unsubstituted or substituted 4- to 8-membered non-aromatic heterocyclyl group an unsubstituted or substituted C₃₋₈ cycloalkyl group, an unsubstituted or substituted straight or branched C₁₋₁₀ alkyl;

R⁶ is an substituted or unsubstituted (C₁₋₆)alkyl, (C₃₋₄)cycloalkyl, or 4- to 7-membered non aromatic heterocyclyl group;

R¹¹ is selected from halo, cyano, methyl, trifluoromethyl, methoxy, trifluoromethoxy or SCH₃;

d is 0, 1, 2 or 3;

and pharmaceutically acceptable derivatives thereof wherein the compound is not {1-[2-(3-chloro-phenylamino)-4-trifluoromethyl-pyrimidin-5-ylmethyl]-piperidin-4-yl}-methanol, formate.

In one embodiment R³ is cyclobutyl or cyclopropylmethyl.

In one embodiment R⁶ is isopropyl, cyclopropyl, tert-butyl or trifluoromethyl.

In one embodiment R⁶ is isopropyl, cyclopropyl or trifluoromethyl.

In one embodiment R¹ is hydrogen.

In one particular embodiment R¹ and R² together with N to which they are attached form a 4- to 8-membered non-aromatic heterocyclyl ring which is selected from pyrrolidinyl, morpholinyl, piperizinyl, piperidinyl and tetrahydropyridinyl.

In one particular embodiment when R³ is nonaromatic heterocyclyl it is selected from pyrrolidinyl, tetrahydrofuranyl, tetrahydrothiophenyl, tetrahydrothiophenyl-s-oxide, tetrahydrothiophenyl-s,s-dioxide morpholinyl, piperidinyl, piperazinyl, tetrahydropyranyl, tetrahydrothiopyranyl, thiomorpholinyl, thiomorpholinyl-s,s-dioxide, tetrahydropyridinyl.

When R¹ and R² together with N to which they are attached form a 4- to 8-membered non-aromatic heterocyclyl ring which is substituted, or when R³ is substituted, the substituent or substituents may be selected from: C₁₋₆ alkyl, C₁₋₆ alkyl-OH, C₁₋₆ alkoxy, a hydroxy group, a cyano group, halo or a sulfonyl group, methylsulfonyl, NR^{8a}R^{8b}, NHCOCH₃, (═O), CONHCH₃ and NHSO₂CH₃, C(O)OC₁₋₆alkyl wherein R^{8a} and R^{8b} are as described above. Additionally when R³ is C₁₋₆ straight or branched alkyl it can be substituted by optionally substituted phenyl, wherein the substituents can be selected from halo, hydroxy or cyano.

When R¹ and R² together with N to which they are attached form a 4- to 8-membered non-aromatic heterocyclyl ring which is substituted, or when R³ is substituted there can be 1, 2 or 3 substituents.

In one embodiment compounds of formula (I) can be selected from compounds of formula (Ic):

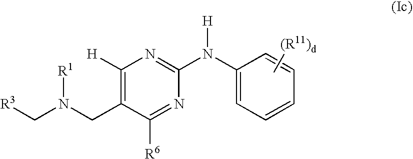

wherein

R¹ is hydrogen or methyl.

R³ is group A, pyridinyl, or pyrimidinyl, any of which can be optionally substituted;

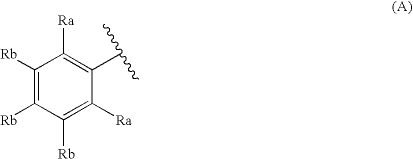

Ra is independently selected from hydrogen, fluoro, chloro or trifluoromethyl;

Rb is independently selected from hydrogen, C₁₋₆ alkyl, C₁₋₆ alkoxy, haloC₁₋₆ alkoxy, hydroxy, cyano, halo, sulfonyl, CONH₂, COOH or NHCOOC₁₋₆alkyl;

R⁶ is an substituted or unsubstituted (C₁₋₆)alkyl, (C₃₋₄)cycloalkyl or 4- to 7-membered non aromatic heterocyclyl group;

R¹¹ is selected from halo, cyano, methyl, trifluoromethyl, methoxy, trifluoromethoxy SCH₃;

d is 0, 1, 2 or 3;

and pharmaceutically acceptable derivatives thereof.

Alternatively R¹ is hydrogen.

When R⁶ is substituted by 1, 2 or 3 substitutents the substituent or substituents may be selected from OK, halo, cyano, C₁₋₆alkoxy, NR^{8a}R^{8b}, NHCOR⁹, NHSO₂R⁹, SOqR⁹; wherein R^{8a}, R^{8b}, R⁹, and q are defined above.

When R¹ and R² taken together with the N to which they are attached form an optionally substituted non-aromatic heterocyclyl ring, the ring may optionally contain 1, 2, 3 or 4 further hetero atoms. The ring may be saturated or unsaturated. In one embodiment the further hetero atoms are selected from oxygen, nitrogen or sulphur. An example of a 4 membered heterocyclyl ring is azetidinyl Examples of 5-membered heterocyclyl rings include pyrrolidinyl, Examples of 6-membered heterocyclyl rings are morpholinyl, piperizinyl, piperidinyl or tetrahydropyridinyl. An additional example is thiomorpholinyl. Examples of a 7-membered heterocyclyl ring are azapine or oxapine. Examples of 8-membered heterocyclyl rings are azacyclooctanyl, azaoxacyclooctanyl or azathiacyclooctanyl.

When R³ or R⁶ is an optionally substituted non-aromatic heterocyclyl group, the ring may contain 1, 2, 3, or 4 hetero atoms. In one embodiment the hetero atoms are selected from oxygen, nitrogen or sulphur. Examples of 4 membered groups are 2- or 3-azetidinyl, oxetanyl, thioxetanyl, thioxetanyl-s-oxide and thioxetanyl-s,s-dioxide. Examples of 5-membered heterocyclyl groups in this instance include dioxalanyl, pyrrolidinyl, tetrahydrofuranyl, tetrahydrothiophenyl, tetrahydrothiophenyl-s-oxide and tetrahydrothiophenyl-s,s-dioxide. Examples of 6-membered heterocyclyl groups are morpholinyl, piperidinyl, piperazinyl, tetrahydropyranyl, tetrahydrothiopyranyl, tetrahydrothiopyranyl-s-dioxide, tetrahydrothiopyranyl-s,s-dioxide, thiomorpholinyl, thiomorpholinyl-s,s-dioxide, tetrahydropyridinyl dioxanyl, and tetrahydro-thiopyran 1,1 dioxide. Examples of a 7-membered heterocyclyl ring are azapine or oxapine. Examples of 8-membered groups are azacyclooctanyl, azaoxacyclooctanyl or azathiacyclooctanyl, oxacylcooctanyl, or thiacyclooctanyl.

When R³ is an unsubstituted or substituted aromatic heterocyclyl group, the ring may contain 1, 2, 3, or 4 hetero atoms. In one embodiment the hetero atoms are selected from oxygen, nitrogen or sulphur. Examples of 5-membered heterocyclyl groups in this instance include furanyl, dioxalanyl, pyrrolyl, oxazolyl, thiazolyl, imidazolyl, oxadiazolyl, thiadiazolyl, triazolyl, triazinyl, isothiazolyl, isoxazolyl, thienyl, pyrazolyl or tetrazolyl. Examples of 6-membered heterocyclyl groups are pyridinyl, pyrizinyl, pyrimidinyl, pyrazinyl, triazinyl, or tetrazinyl.

In one embodiment the compounds of formula (I) are selective for CB2 over CB1. In one embodiment the compounds are 100 fold selective i.e. compounds of formula (I) have an EC50 value at the cloned human cannabinoid CB2 receptor of at least 100 times the EC50 values at the cloned human cannabinoid CB 1 receptor or have less than 10% efficacy at the CB1 receptor. The invention is described using the following definitions unless otherwise indicated.

The term “pharmaceutically acceptable-derivative” means any pharmaceutically acceptable salt, ester, salt of such ester or solvate of the compounds of formula (I), or any other compound which upon administration to the recipient is capable of providing (directly or indirectly) a compound of formula (I) or an active metabolite or residue thereof.

It will be appreciated by those skilled in the art that compounds of formula (I) may be modified to provide pharmaceutically acceptable derivatives thereof at any of the functional groups in the compounds, and that the compounds of formula (I) may be derivatised at more than one position.

It will be appreciated that, for pharmaceutical use, the salts referred to above will be physiologically acceptable salts, but other salts may find use, for example in the preparation of compounds of formula (I) and the physiological acceptable salts thereof. Pharmaceutically acceptable salts include those described by Berge, Bighley and Monkhouse, J. Pharm. Sci., 1977, 66, 1-19. The term “pharmaceutically acceptable salts” includes salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, trishydroxylmethyl amino methane, tripropyl amine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Suitable examples of pharmaceutically acceptable salts include the ammonium, calcium, magnesium, potassium, and sodium salts, and those formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The terms ‘halogen or halo’ are used to represent fluorine, chlorine, bromine or iodine.

The term ‘alkyl’ as a group or part of a group means a straight or branched chain alkyl group or combinations thereof, for example a methyl, ethyl, n-propyl, i-propyl, n-butyl, s-butyl, t-butyl, pentyl, hexyl, 1,1-dimethylethyl, or combinations thereof.

The term ‘alkoxy’ as a group or as part of a group means a straight, branched or cyclic chain alkyl group having an oxygen atom attached to the chain, for example a methoxy, ethoxy, n-propoxy, i-propoxy, n-butoxy, s-butoxy, t-butoxy group, pentoxy, hexyloxy group, cyclopentoxy or cyclohexyloxy group.

The term ‘cycloalkyl’ means a closed 4- to 8-membered non-aromatic ring, for example cyclobutyl, cyclopentyl, cyclohexyl or cycloheptyl, or cyclooctyl.

The term “cycloalkenyl” as a group or part of a group means a non-aromatic ring, containing at least one CH═CH moiety for example cyclobutenyl, cyclopentenyl, cyclohexenyl or cycloheptenyl, or cyclooctenyl.

The term ‘aryl’ means a 5- or 6-membered aromatic ring, for example phenyl, or a 7- to 12-membered bicyclic ring system where at least one of the rings is aromatic, for example naphthyl.

Compounds of formula (I) when R¹² is H can be prepared as set forth in the following scheme:

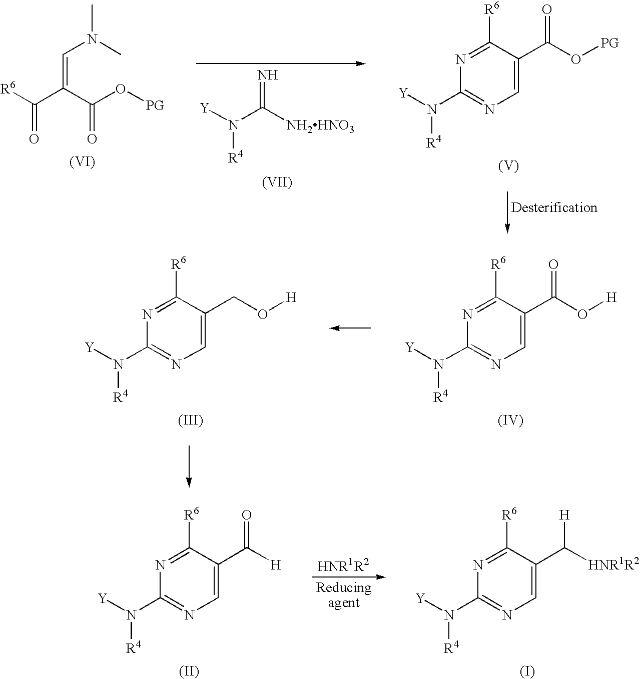

wherein PG is a protecting group for example methyl, ethyl or benzyl, and R¹, R², R⁴, R⁶, and Y are as defined for compounds of formula (I).

Compounds of formula (I) where R¹² is other than hydrogen can be prepared by the following scheme from compounds of formula (II) (prepared as set out in scheme 1):

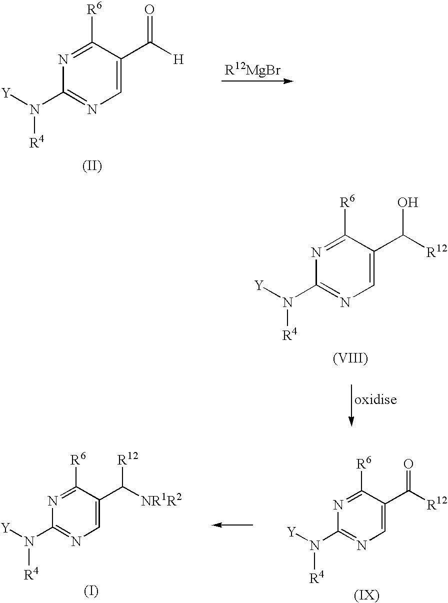

wherein R¹, R², R⁴, R⁶, R¹² and Y are as defined for compounds of formula (I) except R¹² is not hydrogen.

It is to be understood that the present invention encompasses all isomers of compounds of formula (I) and their-pharmaceutically-acceptable derivatives, including all geometric, tautomeric and optical forms, and mixtures thereof (e.g. racemic mixtures). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The subject invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage-requirements and, hence, may be preferred in some circumstances. Isotopically labeled compounds of formula I and following of this invention can generally be prepared by carrying out the procedures disclosed in the Schemes and/or in the Examples below, by substituting a readily available isotopically labeled reagent for a non-isotopically labeled reagent.

The compounds of formula (I) may be prepared in crystalline or non-crystalline form; and, if crystalline, may optionally be hydrated or solvated. This invention includes within its scope stoichiometric hydrates or solvates as well as compounds containing variable amounts of water and/or solvent.

The compounds of the invention may bind selectively to the CB2 receptor, may therefore be useful in treating CB2 receptor mediated diseases.

In view of their ability to bind to the CB2 receptor, the compounds of the invention may be useful in the treatment of the disorders that follow. Thus, the compounds of formula (I) may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

The compounds of the invention may also be useful disease modification or joint structure preservation in multiple sclerosis, rheumatoid arthritis, osteo-arthritis, rheumatoid spondylitis, gouty arthritis and juvenile arthritis.

The compounds of the invention may be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of formula (I) may also be useful in the treatment of fever.

The compounds of formula (I) may also be useful in the treatment of inflammation, for example in the treatment of skin-conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastroesophageal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, polymyositis, gingivitis, myocardial ischemia, pyrexia, systemic lupus erythematosus, tendinitis, bursitis, and Sjogren's syndrome.

The compounds of formula (I) may also be useful in the treatment of bladder hyperrelexia following bladder inflammation.

The compounds of formula (I) may also be useful in the treatment of immunological diseases such as autoimmune diseases, immunological deficiency diseases or organ transplantation. The compounds of formula (I) may also be effective in increasing the latency of HIV infection.

The compounds of formula (I) may also be useful in the treatment of diseases of abnormal platelet function (e.g. occlusive vascular diseases).

The compounds of formula (I) may also be useful in the treatment of neuritis, heart burn, dysphagia, pelvic hypersensitivity, urinary incontinence, cystitis or pruritis.

The compounds of formula (I) may also be useful for the preparation of a drug with diuretic action.

The compounds of formula (I) may also be useful in the treatment of impotence or erectile dysfunction.

The compounds of formula (I) may also be useful for attenuating the hemodynamic side effects of non-steroidal anti-inflammatory drugs (NSAID's) and cyclooxygenase-2 (COX-2) inhibitors.

The compounds of formula (I) may also be useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease; metabolism; toxins; anoxia and vitamin deficiency; and mild cognitive impairment associated with ageing, particularly Age Associated Memory Impairment. The compounds may also be useful for the treatment of amyotrophic lateral sclerosis (ALS) and neuroinflamation.

The compounds of formula (I) may also be useful in neuroprotection and in the treatment of neurodegeneration following stroke, cardiac arrest, pulmonary bypass, traumatic brain injury, spinal cord injury or the like.

The compounds of formula (I) may also be useful in the treatment of tinnitus.

The compounds of formula (I) may also be useful in the treatment of psychiatric disease for example schizophrenia, depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, schizoaffective disorder or the depressed type, and depressive disorders resulting from general medical conditions including, but not limited to, myocardial infarction, diabetes, miscarriage or abortion, etc), anxiety disorders (including generalised anxiety disorder and social anxiety disorder), panic disorder, agoraphobia, social phobia, obsessive compulsive disorder and post-traumatic stress disorder, memory disorders, including dementia, amnesic disorders and age-associated memory impairment, disorders of eating behaviours, including anorexia nervosa and bulimia nervosa, sexual dysfunction, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), amphetamine or amphetamine-related drugs (e.g. dextroamphetamine, methylamphetamine) or a combination thereof.

The compounds of formula (I) may also be useful in preventing or reducing dependence on, or preventing or reducing tolerance or reverse tolerance to, a dependence—inducing agent. Examples of dependence inducing agents include opioids (e.g. morphine), CNS depressants (e.g. ethanol), psychostimulants (e.g. cocaine) and nicotine.

The compounds of formula (I) may also be useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

The term “treatment” or “treating” as used herein includes the treatment of established disorders and also includes the prophylaxis thereof. The term “prophylaxis” is used herein to mean preventing symptoms in an already afflicted subject or preventing recurrance of symptoms in an afflicted subject and is not limited to complete prevention of an afflication.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the activity of cannabinoid 2 receptors.

According to a further aspect of the invention, we provide a method of treating a mammal including a human subject suffering from a condition which is mediated by the activity of cannabinoid 2 receptors which comprises administering to said subject a therapeutically effective amount of a compound of formula (I) or a pharmaceutically acceptable derivative thereof.

According to a further aspect of the invention we provide a method of treating a mammal including a human subject suffering from an immune disorder, an inflammatory disorder, pain, rheumatoid arthritis, multiple sclerosis, osteoarthritis or osteoporosis which method comprises administering to said subject an effective amount of a compound of formula (I) or a pharmaceutically acceptable derivative thereof.

In one embodiment the pain is selected from inflammatory pain, viseral pain, cancer pain, neuropathic pain, lower back pain, muscular skeletal, post operative pain, acute pain and migraine. For example, the inflammatory pain is pain associated with rheumatoid arthritis or osteoarthritis.

According to one aspect of the invention there is provided a compound of formula (I) or a pharmaceutically acceptable derivative thereof for use as a medicament in the treatment of pain.

According to another aspect of the invention there is provided the use of a compound of formula (I) or a pharmaceutically acceptable derivative thereof for the manufacture of a therapeutic agent for the treatment or prevention of a condition such as an immune disorder, an inflammatory disorder, pain, rheumatoid arthritis, multiple sclerosis, osteoarthritis or osteoporosis.

In order to use a compound of formula (I) or a pharmaceutically acceptable derivative thereof for the treatment of humans and other mammals it is normally formulated in accordance with standard pharmaceutical practice as a pharmaceutical composition. Therefore in another aspect of the invention is provided a pharmaceutical composition comprising a compound of formula (I) or a pharmaceutically acceptable derivative thereof adapted for use in human or veterinary medicine.

As used herein, “modulator” means both antagonist, partial or full agonist and inverse agonist. In one embodiment the present modulators are agonists.

Compounds of formula (I) and their pharmaceutically acceptable derivatives may be administered in a standard manner for the treatment of the indicated diseases, for example orally, parentarally, sub-lingually, dermally, intranasally, transdermally, rectally, via inhalation or via buccal administration.

Compounds of formula (I) and their pharmaceutically acceptable derivatives which are active when given orally can be formulated as liquids, tablets, capsules and lozenges. A liquid formulation will generally consist of a suspension or solution of the compound or salt in a liquid carrier for example, ethanol, olive oil, glycerine, glucose (syrup) or water with a flavouring, suspending, or colouring agent. Where the composition is in the form of a tablet, any pharmaceutical carrier-routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers or a semi solid e.g. mono di-glycerides of capric acid, Gelucire™ and Labrasol™, or a hard capsule shell e.g gelatin. Where the composition is in the form of a soft shell capsule e.g. gelatin, any pharmaceutical carrier routinely used for preparing dispersions or suspensions may be considered, for example aqueous gums or oils, and are incorporated in a soft capsule shell.

Typical parenteral compositions consist of a solution or suspension of a compound or derivative in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of formula (I) or a pharmaceutically acceptable derivative thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

In one embodiment the composition is in unit dosage form, for example a tablet, capsule or metered aerosol dose, so that the patient may administer a single dose.

Each dosage unit for oral administration contains suitably from 0.01 mg to 500 mg/Kg, and for example, from 0.01 mg to 100 mg/Kg, and each dosage unit for parenteral administration contains suitably from 0.001 mg to 100 mg/Kg, of a compound of formula (I) or a pharmaceutically acceptable derivative thereof calculated as the free acid. Each dosage unit for intranasal administration contains suitably 1-400 mg and for example, 10 to 200 mg per person. A topical formulation contains suitably 0.01 to 5.0% of a compound of formula (I).

The daily dosage regimen for oral administration is suitably about 0.01 mg/Kg to 11000 mg/Kg, of a compound of formula (I) or a pharmaceutically acceptable derivative thereof calculated as the free acid. The daily dosage regimen for parenteral administration is suitably about 0.001 mg/Kg to 200 mg/Kg, of a compound of formula (I) or a pharmaceutically acceptable derivative thereof calculated as the free acid. The daily dosage regimen for intranasal administration and oral inhalation is suitably about 10 to about 500 mg/person. The active ingredient may be administered from 1 to 6 times a day, sufficient to exhibit the desired activity.

It may be advantageous to prepare the compounds of the present invention as nanoparticles. This may improve the oral bioavailability of the compounds. For the purposes of the present invention “nanoparticulate” is defined as solid particles with 50% of the particles having a particle size of less than 1 μm, for example less than 0.75 μm

The particle size of the solid particles of compound (I) may be determined by laser diffraction. A suitable machine for determining particle size by laser diffraction is a Lecotrac laser particle size analyser, using an HELOS optical bench fitted with a QUIXEL dispersion unit.

Numerous processes for the synthesis of solid particles in nanoparticulate form are known. Typically these processes involve a milling process, for example a wet milling process in the presence of a surface modifying agent that inhibits aggregation and/or crystal growth of the nanoparticles once created. Alternatively these processes may involve a precipitation process, for example, a process of precipitation in an aqueous medium from a solution of the drug in a non-aqueous solvent.

Accordingly, in a further aspect, the present invention provides a process for preparing compound (I) in nanoparticulate form as hereinbefore defined, which process comprises milling or precipitation.

Representative processes for the preparation of solid particles in nanoparticulate form are described in the patents and publications listed below.

U.S. Pat. No. 4,826,689 to Violanto & Fischer, U.S. Pat. No. 5,145,684 to Liversidge et al U.S. Pat. No. 5,298,262 to Na & Rajagopalan, U.S. Pat. No. 5,302,401 Liversidge et al U.S. Pat. No. 5,336,507 to Na & Rajagopalan, U.S. Pat. No. 5,340,564 to Illig & Sarpotdar U.S. Pat. No. 5,346,702 to Na Rajagopalan, U.S. Pat. No. 5,352,459 to Hollister et al U.S. Pat. No. 5,354,560 to Lovrecich, U.S. Pat. No. 5,384,124 to Courteille et al, U.S. Pat. No. 5,429,824 to June, U.S. Pat. No. 5,503,723 to Ruddy et al, U.S. Pat. No. 5,510,118 to Bosch et al, U.S. Pat. No. 5,518 to Bruno et al, U.S. Pat. No. 5,518,738 to Eickhoff et al, U.S. Pat. No. 5,534,270 to De Castro, U.S. Pat. No. 5,536,508 to Canal et al, U.S. Pat. No. 5,552,160 to Liversidge et al, U.S. Pat. No. 5,560,931 to Eickhoff et al, U.S. Pat. No. 5,560,932 to Bagchi et al, U.S. Pat. No. 5,565,188 to Wong et al, U.S. Pat. No. 5,571,536 to Eickhoff et al, U.S. Pat. No. 5,573,783 to Desieno & Stetsko, U.S. Pat. No. 5,580,579 to Ruddy et al, U.S. Pat. No. 5,585,108 to Ruddy et al, U.S. Pat. No. 5,587,143 to Wong, U.S. Pat. No. 5,591,456 to Franson et al, U.S. Pat. No. 5,622,938 to Wong, U.S. Pat. No. 5,662,883 to Bagchi et al, U.S. Pat. No. 5,665,331 to Bagchi et al, U.S. Pat. No. 5,718,919 to Ruddy et al, U.S. Pat. No. 5,747,001 to Wiedmann et al, WO93/25190, WO96/24336, WO 97/14407, WO 98/35666, WO 99/65469, WO 00/18374, WO 00/27369, WO 00/30615 and WO 01/41760.

Such processes may be readily adapted for the preparation of compound (I) in nanoparticulate form. Such processes form a further aspect of the invention.

The process of the present invention for example, uses a wet milling step carried out in a mill such as a dispersion mill in order to produce a nanoparticulate form of the compound. The present invention may be put into practice using a conventional wet milling technique, such as that described in Lachman et al., The Theory and Practice of Industrial Pharmacy, Chapter 2, “Milling” p. 45 (1986).

In a further refinement, WO02/00196 (SmithKline Beecham plc) describes a wet milling procedure using a mill in which at least some of the surfaces are made of nylon (polyamide) comprising one or more internal lubricants, for use in the preparation of solid particles of a drug substance in nanoparticulate form.

In another aspect the present invention provides a process for preparing compounds of the invention in nanoparticulate form comprising wet milling a suspension of compound in a mill having at least one chamber and agitation means, said chamber(s) and/or said agitation means comprising a lubricated nylon, as described in WO02/00196.

The suspension of a compound of the invention for use in the wet milling is typically a liquid suspension of the coarse compound in a liquid medium. By “suspension” is meant that the compound is essentially insoluble in the liquid medium. Representative liquid media include an aqueous medium. Using the process of the present invention the average particle size of coarse compound of the invention may be up to 1 mm in diameter. This advantageously avoids the need to preprocess the compound.

In a further aspect of the invention the aqueous medium to be subjected to the milling comprises compound (I) present in from about 1% to about 40% w/w, for example, from about 10% to about 30% w/w, more In one embodiment about 20% w/w.

The aqueous medium may further comprise one or more pharmaceutically acceptable water-soluble carriers which are suitable for steric stabilisation and the subsequent processing of compound (I) after milling to a pharmaceutical composition, e.g. by spray drying. Pharmaceutically acceptable excipients most suitable for steric stabilisation and spray-drying are surfactants such as poloxamers, sodium lauryl sulphate and polysorbates etc; stabilisers such as celluloses e.g. hydroxypropylmethyl cellulose; and carriers such as carbohydrates e.g. mannitol.

In a further aspect of the invention the aqueous medium to be subjected to the milling may further comprise hydroxypropylmethyl cellulose (HPMC) present from about 0.1 to about 10% w/w. The process of the present invention may comprise the subsequent step of drying compound of the invention to yield a powder.

Accordingly, in a further aspect, the present invention provides a process for preparing a pharmaceutical composition containing a compound of the present invention which process comprises producing compound of formula (I) in nanoparticulate form optionally followed by drying to yield a powder.

A further aspect of the invention is a pharmaceutical composition comprising a compound of formula (I) or a pharmaceutically acceptable deriviate thereof in which the compound of formula (I) or a pharmaceutically acceptable deriviate thereof is present in solid particles in nanoparticulate form, in admixture with one or more pharmaceutically acceptable carriers or excipients.

By “drying” is meant the removal of any water or other liquid vehicle used during the process to keep compound of formula (I) in liquid suspension or solution. This drying step may be any process for drying known in the art, including freeze drying, spray granulation or spray drying. Of these methods spray drying is particularly preferred. All of these techniques are well known in the art. Spray drying/fluid bed granulation of milled compositions is carried out most suitably using a spray dryer such as a Mobile Minor Spray Dryer [Niro, Denmark], or a fluid bed drier, such as those manufactured by Glatt, Germany.

In a further aspect the invention provides a pharmaceutical composition as hereinbefore defined, in the form of a dried powder, obtainable by wet milling solid particles of compound of formula (I) followed by spray-drying the resultant suspension.

In one embodiment, the pharmaceutical composition as hereinbefore defined, further comprises HPMC present in less than 15% w/w, for example, in the range 0.1 to 10% w/w.

The CB₂ receptor compounds for use in the instant invention may be used in combination with other therapeutic agents, for example COX-2 inhibitors, such as celecoxib, deracoxib, rofecoxib, valdecoxib, parecoxib or COX-189; 5-lipoxygenase inhibitors; NSAID's, such as aspirin, diclofenac, indomethacin, nabumetone or ibuprofen; leukotriene receptor antagonists; DMARD's such as methotrexate; adenosine A1 receptor agonists; sodium channel blockers, such as lamotrigine; NMDA receptor modulators, such as glycine receptor antagonists; gabapentin and related compounds; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; EP, receptor ligands, EP₄ receptor ligands; EP₂ receptor ligands; EP₃ receptor ligands; EP₄ antagonists; EP₂ antagonists and EP₃ antagonists; bradykinin receptor ligands and vanilloid receptor ligand, antirheumatoid arthritis drugs, for example anti TNF drugs e.g. enbrel, remicade, anti-IL-1 drugs, or DMARDS e.g. leflunamide. When the compounds are used in combination with other therapeutic agents, the compounds may be administered either sequentially or simultaneously by any convenient route.

Additional COX-2 inhibitors are disclosed in U.S. Pat. Nos. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

The compounds of the present invention may be administered in combination with other active substances such as 5HT3 antagonists, NK-1 antagonists, serotonin agonists, selective serotonin reuptake inhibitors (SSRI), noradrenaline re-uptake inhibitors (SNRI), tricyclic antidepressants and/or dopaminergic antidepressants.

Suitable 5HT3 antagonists which may be used in combination of the compound of the inventions include for example ondansetron, granisetron, metoclopramide.

Suitable serotonin agonists which may be used in combination with the compound of the invention include sumatriptan, rauwolscine, yohimbine, metoclopramide.

Suitable SSRIs which may be used in combination with the compound of the invention include fluoxetine, citalopram, femoxetine, fluvoxamine, paroxetine, indalpine, sertraline, zimeldine.

Suitable SNRIs which may be used in combination with the compound of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Compounds of the present invention may be used in combination with PDE4 inhibitors. The PDE4 inhibitor useful in this invention may be any compound that is known to inhibit the PDE4 enzyme or which is discovered to act in as PDE4 inhibitor, and which is only or essentially only a PDE4 inhibitor, not compounds which inhibit to a degree of exhibiting a therapeutic effect other members of the PDE family as well as PDE4. Generally it is preferred to use a PDE4 antagonist which has an IC₅₀ ratio of about 0.1 or greater as regards the IC₅₀ for the PDE4 catalytic form which binds rolipram with a high affinity divided by the IC₅₀ for the form which binds rolipram with a low affinity. Compounds of the present invention or combinations with PDE4 can be used in treating inflammation and as bronchodilators.

It turns out that there are at least two binding forms on human monocyte recombinant PDE 4 (hPDE 4) at which inhibitors bind. One explanation for these observations is that hPDE 4 exists in two distinct forms. One binds the likes of rolipram and denbufylline with a high affinity while the other binds these compounds with a low affinity. The preferred PDE4 inhibitors of for use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE 4 catalytic form which binds rolipram with a high affinity divided by the IC₅₀ for the form which binds rolipram with a low affinity.

Reference is made to U.S. Pat. No. 5,998,428, which describes these methods in more detail. It is incorporated herein in full as though set forth herein.

Most suitable are PDE4 inhibitors which have an IC₅₀ ratio of greater than 0.5, and particularly those compounds having a ratio of greater than 1.0.

A further aspect of the invention is an CB2 modulator in combination with a PDE4 inhibitor and pharmaceutical compositions comprising said combination.

A further aspect of the invention is a method of treating lung disorders for example asthma, bronchitis, emphysema, allergic rhinitis, respiratory distress syndrome, pigeon fancier's disease, farmer's lung, chronic obstructive pulmonary disease, (COPD) and cough or a disorder which can be treated with a broncodilator which comprises administering to a mammal including man, an effective amount of a CB modulator or a pharmaceutically acceptable derivative therefore and an effective amount of a PDE4 inhibitor or a pharmaceutically acceptable derivative thereof.

An additional aspect of the invention is the use of an effective amount of a CB2 modulator or a pharmaceutically acceptable derivative therefore and an effective amount of a PDE4 inhibitor or a pharmaceutically acceptable derivative thereof in the manufacture of a medicament in the treatment of lung disorders for example asthma, bronchitis; emphysema, allergic rhinitis, respiratory distress syndrome, pigeon fancier's disease, farmer's lung, chronic obstructive pulmonary disease, (COPD) and cough or for the manufacture of a brocodilator.

When used herein cough can have a number of forms and includes productive, non-productive, hyper-reactive, asthma and COPD associated.

A further aspect of the invention is a patient pack comprising an effective amount of a CB2 modulator or a pharmaceutically acceptable derivative therefore and an effective amount of a PDE4 inhibitor or a pharmaceutically acceptable derivative

Suitable PDE4 compounds are cis [cyano-4-(3-cyclopentyloxy-4-methoxyphenyl)cyclohexan-1-carboxylate] also known as cilomilast or Ariflo®, 2-carbomethoxy-4-cyano-4-(3-cyclopropylmethoxy-4-difluoromethoxyphenyl)cyclohexan-1-one, and cis [4-cyano-4-(3-cyclopropylmethoxy-4-difluoromethoxyphenyl)cyclohexan-1-ol]. They can be made by the processed described in U.S. Pat. Nos. 5,449,686 and 5,552,438. Other PDE4 inhibitors, specific inhibitors, which can be used in this invention are AWD-12-281 from ASTA MEDICA (Hofgen, N. et al. 15th EFMC Int Symp Med Chem (September 6-10, Edinburgh) 1998, Abst P. 98); a 9-benzyladenine derivative nominated NCS-613 (INSERM); D-4418 from Chiroscience and Schering-Plough; a benzodiazepine PDE4 inhibitor identified as CI-1018 (PD-168787; Parke-Davis/Warner-Lambert); a benzodioxole derivative Kyowa Hakko disclosed in WO 9916766; V-11294A from Napp (Landells, L. J. et al. Eur Resp J [Annu Cong Eur Resp Soc (September 19-23, Geneva) 1998] 1998, 12(Suppl. 28): Abst P2393); roflumilast (CAS reference No 162401-32-3) and a pthalazinone (WO 99/47505) from Byk-Gulden (now Altana); or a compound identified as T-440 (Tanabe Seiyaku; Fuji, K. et al. J Pharmacol Exp Ther, 1998, 284(1): 162).

Additional PDE4 inhibitors are disclosed on pages 2 to 15 of WO01/13953. Specifically selected are arofylline, atizoram, BAY-19-8004, benafentrine, BYK-33043, CC-3052, CDP-840, cipamfylline, CP-220629, CP-293121, D-22888, D-4396, denbufylline, filaminast, GW-3600, ibudilast, KF-17625, KS-506-G, laprafylline, NA-0226A, NA-23063A, ORG-20241, ORG-30029, PDB-093, pentoxifylline, piclamilast, rolipram, RPR-117658, RPR-122818, RPR-132294, RPR-132703, RS-17597, RS-25344-000, SB-207499, SB210667, SB211572, SB-211600, SB212066, SB212179, SDZ-ISQ-844, SDZ-MNS-949, SKF-107806, SQ-20006, T-2585, tibenelast, tolafentrine, UCB-29646, V-11294A, YM-58997, YM-976 and zardaverine. In one embodiment the PDE4 inhibitor is selected from cilomilast, AWD-12-281, NCS-613, D-4418, CI-1018, V-11294A, roflumilast or T-440.

It will be appreciated that the compounds of any of the above combinations or compositions may be administered simultaneously (either in the same or different pharmaceutical formulations), separately or sequentially.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

All publications, including but not limited to patents and patent applications, cited in this specification are herein incorporated by reference as if each individual publication were specifically and individually indicated to be incorporated by reference herein as though fully set forth.

### Determination of Cannabinoid CB1 Receptor Agonist Activity

The cannabinoid CB1 receptor agonist activity of the compounds of formula (I) was determined in accordance with the following experimental method.

### Experimental Method

Yeast (Saccharomyces cerevisiae) cells expressing the human cannabinoid CB1 receptor were generated by integration of an expression cassette into the ura3 chromosomal locus of yeast strain MMY23. This cassette consisted of DNA sequence encoding the human CB1 receptor flanked by the yeast GPD promoter to the 5′ end of CB 1 and a yeast transcriptional terminator sequence to the 3′ end of CB1. MMY23 expresses a yeast/mammalian chimeric G-protein alpha subunit in which the C-terminal 5 amino acids of Gpal are replaced with the C-terminal 5 amino acids of human Gαi3 (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 mM stocks in DMSO. EC₅₀ values (the concentration required to produce 50% maximal response) were estimated using dilutions of between 3- and 5-fold (BiomekFX, Beckman) into DMSO. Agonist solutions in DMSO (1% final assay volume) were transferred into black, clear bottom, microtitre plates from NUNC (96- or 384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 20 μM fluorescein di-β-D-glucopyranoside (FDGlu). This mixture (50 ul per well for 384-well plates, 200 ul per well for 96-well plates) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a Spectrofluor microtitre plate reader (Tecan; excitation wavelength: 485 nm; emission wavelength: 535 nm). Fluorescence was plotted against compound concentration and iteratively curve fitted using a four parameter fit to generate a concentration effect value. Efficacy (Eₘₐₓ) was calculated from the equation

Eₘₐₓ=Max_{[compound X]}−Min_{[compound X]}/Max_{[HU210]}−Min_{[HU210]}×100%

where Max_{[compound X]} and Min_{[compound X]} are the fitted maximum and minimum respectively from the concentration effect curve for compound X, and Max_{[HU210]} and Min_{[HU210]} are the fitted maximum and minimum respectively from the concentration effect curve for (6aR,10aR)-3-(1,1′-Dimethylheptyl)-6a,7,10,10a-tetrahydro-1 hydroxy-6,6-dimethyl-6H-dibenzo[b,d]pyran-9-methanol (HU210; available from Tocris). Equieffective molar ratio (EMR) values were calculated from the equation

EMR=EC_{50[compound X]}/EC_{50[HU210]}

Where EC_{50[compound X]} is the EC₅₀ of compound X and EC_{50[HU210]} is the EC₅₀ of HU210.

Compounds of the Examples tested according to this method had EC50 values>1,000 nM and/or efficacy values<50% at the cloned human CB1 receptor, except for Examples 89 and 91, which had EC50 values of 300-1000 nM and efficacies of between 50-100% at the cloned human CB1 receptor

### Determination of Cannabinoid CB2 Receptor Agonist Activity

The cannabinoid CB2 receptor agonist activity of the compounds of formula (I) was determined in accordance with the following experimental method.

### Experimental Method

Yeast (Saccharomyces cerevisiae) cells expressing the human cannabinoid CB2 receptor were generated by integration of an expression cassette into the ura3 chromosomal locus of yeast strain MMY23. This cassette consisted of DNA sequence encoding the human CB2 receptor flanked by the yeast GPD promoter to the 5′ end of CB2 and a yeast transcriptional terminator sequence to the 3′ end of CB2. MMY23 expresses a yeast/mammalian chimeric G-protein alpha subunit in which the C-terminal 5 amino acids of Gpa1 are replaced with the C-terminal 5 amino acids of human Gαi3 (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 mM stocks in DMSO. EC₅₀ values (the concentration required to produce 50% maximal response) were estimated using dilutions of between 3- and 5-fold (BiomekFX, Beckman) into DMSO. Agonist solutions in DMSO (1% final assay volume) were transferred into black, clear bottom, microtitre plates from NUNC (96- or 384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 20M fluorescein di-β-D-glucopyranoside (FDGlu). This mixture (50 ul per well for 384-well plates, 200 ul per well for 96-well plates) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a Spectrofluor microtitre plate reader (Tecan; excitation wavelength: 485 nm; emission wavelength: 535 nm). Fluorescence was plotted against compound concentration and iteratively curve fitted using a four parameter fit to generate a concentration effect value. Efficacy (Eₘₐₓ) was calculated from the equation

Eₘₐₓ=Max_{[compound X]}−Min_{[compound X]}/Max_{[HU210]}−Min_{[HU210]}×100%

where Max_{[compound X]} and Min_{[compound X]} are the fitted maximum and minimum respectively from the concentration effect curve for compound X, and Max_{[HU210]} and Min_{[HU210]} are the fitted maximum and minimum respectively from the concentration effect curve for (6aR,10aR)-3-1,1′-Dimethylheptyl)-6a,7,10,10a-tetrahydro-1-hydroxy-6,6-dimethyl-6H-dibenzo[b,d]pyran-9-methanol (HU210; available from Tocris). Equieffective molar ratio (EMR) values were calculated from the equation

EMR=EC_{50[compound X]}/EC_{50[HU210]}

Where EC_{50[compound X]} is the EC₅₀ of compound X and EC_{50[HU210]} is the EC₅₀ of HU210.

The compounds of Example 1 to 11, 13 to 38 and 85 to 95 and 97 to 103 tested according to this method had an EC₅₀ values of <300 nM and efficacy value of >50% at the cloned human cannabinoid CB2 receptor.

The compounds of Examples 12, 39 to 59 and 96 tested according to this method had an EC₅₀ values between 300 nM and 1000 nM and efficacy value of >50% at the cloned human cannabinoid CB2 receptor.

The compounds of Example 60 to 82, 104 and 105 tested according to this method had an EC₅₀ values between >1000 nM and/or an efficacy value of <50% at the cloned human cannabinoid CB2 receptor.

The compounds of Examples 83 and 84 had no appreciable potency or efficacy at the CB2 receptor.

The following examples are illustrative, but not limiting of the embodiments of the present invention.
Conditions, Hardware, and Software used for Mass-directed Autopurification Hardware
Waters 600 gradient pump, Waters 2700 sample manager, Waters Reagent Manager, Micromass ZMD mass spectrometer, Gilson 202—fraction collector, Gilson Aspec—waste collector.

### Software

Micromass Masslynx version 3.5

### Column

The column used is typically a Supelco ABZ+ column whose dimensions are 10 mm internal diameter by 100 mm in length. The stationary phase particle size is 5 μm.

### Solvents

A. Aqueous solvent=Water+0.1% Formic Acid
B. Organic solvent=MeCN:Water 95:5+0.05% Formic Acid
Make up solvent=MeOH:Water 80:20+50 mMol Ammonium Acetate
Needle rinse solvent=MeOH:Water:DMSO 80:10:10

### Methods

Five methods are used depending on the analytical retention time of the compound of interest. They all have a flow rate of 20 ml/min and a 15-minute runtime, which comprises of a 10-minute gradient followed by a 5-minute column flush and re-equilibration step.

### Method 1 MDP 1.5-2.2=0-30% B

### Method 2 MDP 2.0-2.8=5-30% B

### Method 3 MDP 2.5-3.0=15-55% B

### Method 4 MDP 2.8-4.0=30-80% B

### Method 5 MDP 3.8-5.5=50-90% B

### Conditions Used for Analytical LCMS Systems

### Hardware

Agilent 1100 gradient pump

### Agilent 1100 Autosampler

### Agilent 1100 PDA Dectector

### Agilent 1100 Degasser

Micromass ZQ mass spectrometer

### PL-ELS 1000

### Software

Micromass Masslynx versions 3.5/4.0

### Column

The column used is a Supelcosil ABZ+PLUS, the dimensions of which are 4.6 mm×33 mm. The stationary phase particle size is 3 m.

### Solvents

A: Aqueous solvent=10 mMol Ammonium Acetate+0.1% Formic Acid
B: Organic solvent=95% Acetonitrile+0.05% Formic Acid

### Method

The generic method used has 5.5 minute runtime, which comprises of a 4.7-minute gradient (0-100% B) followed by a 0.6 minute column flush and 0.2 minute re-equilibration step.

### Flow Rate

The above method has a flow rate of 3 ml/mins

### Conditions Used for NMR

### Hardware

### Bruker 400 MHz Ultrashield

### Bruker B-ACS60 Autosampler

### Bruker Advance 400 Console

### Software

User interface—NMR Kiosk
Controlling software—XWin NMR version 3.0

### Conditions Used for the Biotage Horizon.

### Column: Biotage C18HS 25+S

Fraction volume: 9 ml • UV Threshold: 0.03 AU

### Solvent A=Water, B=Acetonitrile

Gradient:

| Volume(ml) | A | B |
|---|---|---|
| 0 | 70% | 30% |
| 240 | 0% | 100% |

### Abbreviations:

AcOH (acetic acid), Bn (benzyl), Bu, Pr, Me, Et (butyl, propyl, methyl ethyl), DMSO (dimethyl sulfoxide), DCM (dichloromethane), DME (1,2-dimethoxyethane), DMF (N,N-dimethylformamide), EDC (1-(3-dimethylaminopropyl)-3-ethylcarbodiimide), EtOAc (ethyl acetate), EtOH (ethanol), HPLC (High pressure liquid chromatography), LC/MS (Liquid chromatography/Mass spectroscopy), MDAP (Mass Directed AutoPurification), MeCN (acetonitrile), MeOH (methanol), NMR (Nuclear Magnetic Resonance (spectrum)), NMP (n-methyl pyrrolidone), SPE (Solid Phase Extraction), THF (tetrahydrofuran), s, d, t, q, m, br (singlet, doublet, triplet, quartet, multiplet, broad.)

### Intermediate 1: 2-(3-Chloro-phenylamino)-4-cyclopropyl-pyrimidine-5-carboxylic acid methyl ester

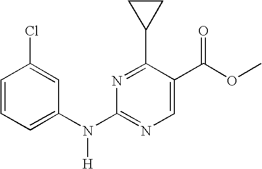

A solution of N-(3-chloro-phenyl)-guanidine nitrate salt (prepared as in WO 95/09851, 5.00 g, 0.0215 mol. 1 eq) in ethanol (100 ml) was stirred with sodium ethoxide (1.48 g, 0.0217 mol, 1.01 eq) for 2 min. (1-Cyclopropyl-methanoyl)dimethylamino-acrylic acid methyl ester (prepared as in EP1101763A2, 4.24 g, 0.0215 mol, 1 eq) was added, and the reaction mixture was heated at 920 for 2 hours. The volatiles were removed in vacuo and the concentrated reaction mixture was diluted with ethyl acetate. The organic layer was washed with water, brine, dried (MgSO₄), filtered, and the volatiles were removed in vacuo. The residue was then purified by chromatography using Biotage Flash 40M cartridge eluting with 10% EtOAc:iso-hexane, increasing to 30% EtOAc:iso-hexane, to yield the title compound (4.14 g, 0.0136 mol, 63%) as an off-white solid.

LC/MS t=3.73 min. Molecular ion observed [MH+] 304, consistent with molecular formula

### Intermediate 2: 2-(3-Chloro-phenylamino)-4-cyclopropyl-pyrimidine-5-carboxylic acid

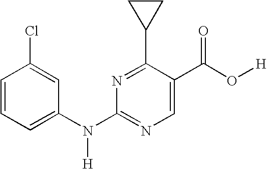

2-(3-Chloro-phenylamino)-4-cyclopropyl-pyrimidine-5-carboxylic acid methyl ester (2.23 g, 0.735 mmol, 1 eq), was dissolved in THF (20 ml) and lithium-hydroxide (0.926 g, 0.022 mol, 3 eq), was added. The reaction mixture was stirred at 21° C. for 48 hours. The volatiles were removed in vacuo and water added. The mixture was acidified to pHi with conc hydrochloric acid, stirred for 2 min, the precipitate was filtered off, and washed with water. The solid was then co-evaporated with toluene to yield the dry title compound (1.00 g, 0.345 mmol, 47%) as a white solid.

LC/MS t=3.77 min. Molecular ion observed [MH⁺] 290, consistent with molecular formula C₁₄H₁₂³⁵ClN₃O₂

### Intermediate 3: [2-(3-Chloro-phenylamino)-4-cyclopropyl-pyrimidin-5-yl]-methanol

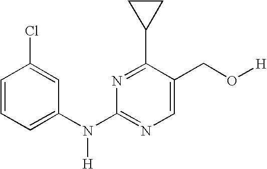

N-methylmorpholine (0.559 ml, 0.497 mmol, 1 eq) and isobutylchloroformate (0.648 ml, 0.497 mmol, 1 eq) were successively added to a stirred solution of 2-(3-chloro-phenylamino)-4-cyclopropyl-pyrimidine-5-carboxylic acid (1.441 g, 0.497 mmol, 1 eq) in 1,2-dimethoxyethane (25 ml) at −15° C. and stirring was continued for 10 min at −15° C. The precipitate was filtered off, discarded, and a solution of NaBH₄ (0.284 g, 0.747 mmol, 1.5 eq) in water (1.5 ml) was added at 0° C. The reaction mixture was stirred for 3 min, quenched with water, and extracted with ethyl acetate. The combined organic extracts were washed with brine, dried (MgSO₄), and evaporated in vacuo to yield the title compound (0.895 g, 0.325 mmol, 65%) as a yellow oil.

LC/MS t=3.22 min. Molecular ion observed [MH⁺] 276, consistent with molecular formula C₁₄H₁₄³⁵ClN₃O

### Intermediate 4: 2-(3-Chloro-phenylamino)-4-cyclopropyl-pyrimidine-5-carbaldehyde

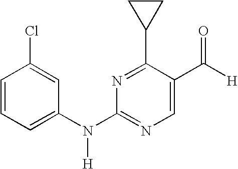

Manganese(IV)oxide (2.757 g, 0.032 mol, 10 eq) and sodium chloride (3.79 g, 0.044 mol, 4 eq) were added to a stirred solution of 2-(3-chloro-phenylamino)-4-cyclopropyl-pyrimidin-5-yl]-methanol (0.923 g, 0.3177 mmol, 1 eq) in dichloromethane (40 ml). After stirring at 21° C. for 18 hours, the precipitate was filtered off, washed with dichloromethane, and dried in vacuo to yield the title compound (0.150 g, 0.055 mmol, 17%) as a pale yellow solid.

LC/MS t=3.56 min. Molecular ion observed [MH⁺] 274, consistent with molecular formula C₁₄H₁₂³⁵ClN₃O.

### Intermediate 5: 2-(3-Chloro-phenylamino)-4-isopropyl-pyrimidine-5-carboxylic acid ethyl ester

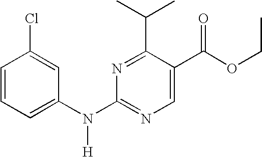

The title compound was prepared as for Intermediate 1 using 1-(isopropyl-methanoyl)-dimethylamino-acrylic acid ethyl ester (prepared in a manner similar to that described by G Mennozi, J Heterocyclic Chem. 1987, 24, 1669)

LC/MS t=3.73 [MH+] 304 Consistent with molecular formula C₁₋₆H₁₈Cl³⁵N₃O₂

### Intermediate 6: 2-(3-Chloro-phenylamino)-4-isopropyl-pyrimidine-5-carboxylic acid

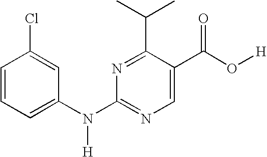

The title compound was prepared as for Intermediate 2 using 2-(3-chloro-phenylamino)-4-isopropyl-pyrimidine-5-carboxylic acid ethyl ester.

LC/MS t=3.81 min. Molecular ion observed [MH⁺] 320, consistent with molecular formula

### Intermediate 7: [2-(3-Chloro-phenylamino)-4-isopropyl-pyrimidin-5-yl]-methanol

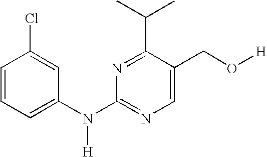

The title compound was prepared from 2-(3-chloro-phenylamino)-4-isopropyl-pyrimidine-5-carboxylic acid using the procedure described Intermediate 3.

LC/MS t=3.28 min. Molecular ion observed [MH+] 278, consistent with molecular formula C₁₄H₁₆³⁵ClN3O.

### Intermediate 8: 2-(3-Chloro-phenylamino)-4-isopropyl-pyrimidine-5-carbaldehyde

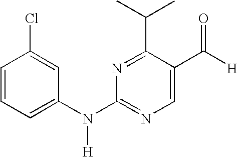

The title compound was prepared from [2-(3-chloro-phenylamino)-4-isopropyl-pyrimidin-5-yl]-methanol using the procedure described for Intermediate 4.

LC/MS t=3.62 min. Molecular ion observed [MH⁺] 276, consistent with molecular formula C₁₄H₁₄³⁵ClN₃O

### Intermediate 9: 2-(3-Chlorophenylamino)-4-trifluoromethyl-pyrimidine-5-carboxylic acid methyl ester

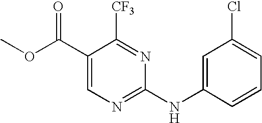

To a solution of methyl 2-chloro-4-trifluoromethyl-pyrimidine-5-carboxylate (11.0 g, ex Maybridge) in 1,4-dioxan (30 ml) was added 3-chloroaniline (17 g) and the solution stirred under reflux for 2 h. 1,4-Dioxan was removed under reduced pressure and the residue stirred in 2N hydrochloric acid (150 ml) for 1 h. The solid was filtered onto a sinter and washed with 2N hydrochloric acid (2×100 ml) and water (5×100 ml). The solid was transferred to a crystallising dish and dried at 50° C. over sodium hydroxide in a vacuum oven (15.2 g)

NMR (DMSO-d6) δ 3.86 (3H, s), 7.15 (1H, dd), 7.39 (1H, t), 7.67 (11, dd), 7.97 (1H, s), 9.10 (1H, s), 10.95 (1H, s).

LC/MS, t=3.69 min, molecular ion observed [MR⁺] 332, consistent with C₁₃H₉ClF₃N₃O₂.

### Intermediate 10: 2-(3-Chlorophenylamino)-4-trifluoromethyl-pyrimidine-5-carboxylic acid

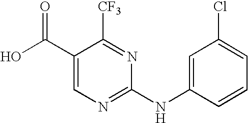

To a suspension of 2-(3-chlorophenylamino)-4-trifluoromethyl-pyrimidine-5-carboxylic acid methyl ester, (15.2 g) in methanol (100 ml) was added a solution of potassium hydroxide (7.68 g) in methanol (100 ml) and the mixture stirred at reflux for 3 h. Methanol was removed under reduced pressure and water (200 ml) added. The solution was washed with ether and concentrated hydrochloric acid was added to adjust the acidity to pH 1. The acidified aqueous was extracted with ethyl acetate (2×200 ml) and the combined extract was washed with water (3×200 ml). The dried (MgSO₄) organic layer was evaporated and the residue triturated with isohexane to afford 2-(3-chlorophenylamino)-4-trifluoromethyl-pyrimidine-5-carboxylic acid (14.35 g).

NMR (DMSO-d6) δ 7.52 (1H, dd), 7.78 (1H, t), 8.07 (1H, dd), 8.38 (1H, s), 9.49 (1H, s), 11.20 (1H, s), 14.50 (1H, s).

LC/MS, t=3.83 min, molecular ion observed [MH⁺] 318, consistent with C₁₂H₇ClF₃N₃O₂

### Intermediate 11: [2-(3-Chlorophenylamino)-4-trifluoromethyl-pyrimidin-5-yl]-methanol

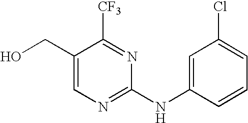

To a solution of 2-(3-chlorophenylamino)-4-trifluoromethyl-pyrimidine-5-carboxylic acid (3.0 g) in 1,2-dimethoxyethane (48 ml) under nitrogen at −15° C. was added N-methylmorpholine (1.05 ml) followed by isobutyl chloroformate (1.22 ml). After 10 minutes, the precipitated N-methylmorpholine hydrochloride was removed by filtration and the filtrate, under nitrogen, was treated with a solution of sodium borohydride (537 mg) in water (5 ml). After a further 5 minutes, the reaction was quenched by the addition of water. The aqueous mixture was extracted with ethyl acetate (3×150 ml), combined, dried (MgSO₄), and evaporated in vacuo. The residue was purified by Biotage flash chromatography over Merck 9385 silica gel eluting with 3:2 isohexane:ethyl acetate to afford [2-(3-chlorophenylamino)-4-trifluoromethyl-pyrimidin-5-yl]-methanol (1.97 g).

NMR (DMSO-d6) δ 4.57 (2H, d), 5.45 (1H, s), 7.04 (1H, dd), 7.33 (1H, t), 7.66 (1H, dd), 7.97 (1H, t), 8.84 (1H, s), 10.30 (1H, s).

LC/MS, t=3.26 min, molecular ion observed [MH⁺] 304, consistent with C₁₂H₉ClF₃N₃O.

### Intermediate 12: 2-(3-Chlorophenylamino)-4-trifluoromethyl-pyrimidine-5-carbaldehyde

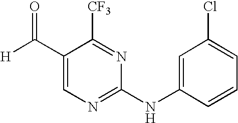

To a solution of [2-(3-chlorophenylamino)-4-trifluoromethyl-pyrimidin-5-yl]-methanol Intermediate 11 (1.97 g) in dichloromethane (81 ml) was added sodium chloride (5.39 g) and manganese (IV) oxide (5.64 g), and the mixture stirred at room temperature overnight. The mixture was filtered onto a bed of Celite washing with dichloromethane. The combined filtrates were evaporated under reduced pressure to afford 2-(3-chlorophenylamino)-4-trifluoromethyl-pyrimidine-5-carbaldehyde (1.76 g).

NMR (DMSO-d6) δ 7.20 (1H, dd), 7.41 (1H, t), 7.69 (1H, dd), 7.99 (1H, s), 9.18 (1H, s), 10.00 (1H, s), 11.10 (1H, s).

LC/MS, t=3.60 min, molecular ion observed [MH⁺] 302, consistent with C₁₂H₇ClF₃N₃O

### Intermediate 13: (5-Bromomethyl-4-trifluoromethyl-pyrimidin-2-yl)-(3-chloro-phenyl)-amine

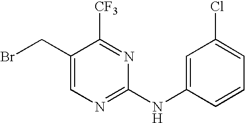

To a solution of [2-(3-chlorophenylamino)-4-trifluoromethyl-pyrimidin-5-yl]-methanol (42 mg), and carbon tetrabromide (183 mg) in tetrahydrofuran (2 ml) was added triphenylphosphine (72 mg). The solution was stirred at room temperature for 1 h and then evaporated under reduced pressure. The residue was dissolved in isohexane:dichloromethane 3:1 and purified by chromatography on a Sep-pak cartridge of silica gel, eluting with isohexane:dichloromethane 2:1 to afford the title compound as a pale yellow solid (27 mg).

NMR (DMSO-d6) δ 4.77 (2H, s), 7.08 (1H, dd), 7.36 (1H, t), 7.65 (1H, dd), 7.95 (1H, s), 8.94 (1H, s), 10.60 (1H, s).

LC/MS, t=3.89 min, molecular ion observed [MH⁺] 368, consistent with C₁₂H₈⁸¹Br³⁵ClF₃N₃.

### Intermediate 14. 2-Amino-4-trifluoromethyl-pyrimidine-5-carboxylic acid

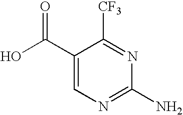

A solution of 2-amino-4-trifluoromethyl-pyrimidine-5-carboxylic acid ethyl ester (commercially available from Maybridge) (14.9 g) in methanol (300 ml) containing potassium hydroxide (10.65 g) was refluxed for 4 h. The solvent was removed under reduced pressure and the residue dissolved in water (150 ml). The aqueous was washed with diethyl ether (75 ml) and then acidified to pH 1 with concentrated hydrochloric acid to afford a white precipitate. The solid was filtered off, washed with water and dried to afford the title compound (12.76 g).

NMR (DMSO-d6) δ 7.94 (2H, s), 8.82 (1H, s), 13.30 (1H, s).

LC/MS, t=1.10 min, molecular ion observed [MH⁺] 208, consistent with C₆H₄F₃N₃O₂.

### Intermediate 15. (2-Amino-4-trifluoromethyl-pyrimidin-5-yl)-methanol

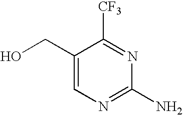

To a solution of 2-amino-4-trifluoromethyl-pyrimidine-5-carboxylic acid (4.35 g) in 1,2-dimethoxyethane (200 ml) at −12° C. was added N-methylmorpholine (2.30 ml) followed by isobutyl chloroformate (2.72 ml). After 5 minutes, the precipitated N-methylmorpholine hydrochloride was removed by filtration and the filtrate treated with a solution of sodium borohydride (1.2 g) in water (10 ml). After a further 30 minutes, the reaction was quenched by the addition of water. The aqueous mixture was extracted with ethyl acetate (3×150 ml), and the combined, dried (MgSO₄) organic extracts were evaporated in vacuo. The residue was purified by Biotage flash chromatography over Merck 9385 silica gel eluting with 1:1 isohexane:ethyl acetate to afford the title compound (1.2 g).

NMR (DMSO-d6) δ 4.45 (2H, d), 5.25 (1H, t), 7.19 (2H, s), 8.52 (1H, s).

LC/MS, t=1.40 min, molecular ion observed [MH⁺] 194, consistent with C₆H₆F₃N₃O.

### Intermediate 16: 2-Amino-4-trifluoromethyl-pyrimidine-5-carbaldehyde

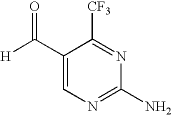

To a solution of (2-amino-4-trifluoromethyl-pyrimidin-5-yl)-methanol (1.2 g) in ethyl acetate (20 ml) was added sodium chloride (5.09 g) and manganese (IV) oxide (5.4 g), and the mixture stirred at room temperature overnight. The mixture was filtered onto a bed of Celite washing with ethyl acetate. The solvent was removed under reduced pressure to afford the title compound (1.16 g).

NMR (DMSO-d6) δ 8.38 (2H, d), 8.92 (1H, s), 9.94 (1H, s).

LC/MS, t=1.80 min, molecular ion observed [M−H⁺] 190, consistent with molecular formula C₆H₄F₃N₃O.

### Intermediate 17: 5-[(Cyclopropylmethyl-amino)-methyl]-4-trifluoromethyl-pyrimidin-2-ylamine

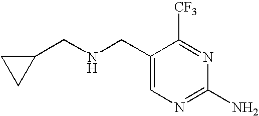

A mixture of 2-amino-4-trifluoromethyl-pyrimidine-5-carbaldehyde (1.16 g), powdered 4A molecular sieves, cyclopropylmethylamine (518 mg) and glacial acetic acid (348 ul) in tetrahydrofuran (20 ml) was stirred at room temperature for 30 min. Sodium triacetoxyborohydride (1.8 g) was added and the mixture stirred overnight. The mixture was diluted with ethyl acetate and washed with 1N NaOH and brine then dried (MgSO₄), filtered and evaporated under reduced pressure to afford the title compound as a pale yellow solid (1.35 g).

NMR (DMSO-d6) δ 0.09 (2H, m), 0.38 (2H, m), 0.85 (1H, m), 2.39 (21, d), 3.68 (2H, s), 7.15 (2H, s), 8.56 (1H, s).

LC/MS, t=1.20 min, molecular ion observed [MH⁺] 247, consistent with molecular formula C₁₀H₁₃F₃N₄.

### Intermediate 18: (2-Amino-4-trifluoromethyl-pyrimidin-5-ylmethyl)-cyclopropylmethyl-carbamic acid dimethyl-ethyl ester

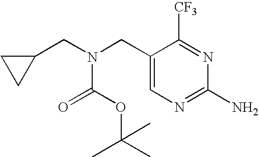

To a solution of 5-[(cyclopropylmethyl-amino)-methyl]-4-trifluoromethyl-pyrimidin-2-ylamine (1.35 g) in ethyl acetate (20 ml), was added triethylamine (0.916 ml) followed by di-tert-butyl dicarbonate (1.31 g) and the solution stirred at room temperature for 3 h. This was diluted with ethyl acetate, washed with water, and the dried (MgSO₄) organic layer was evaporated under reduced pressure. The residue was triturated with hexane to afford the title compound as a white solid (1.56 g).

NMR (DMSO-d6) δ 0.12 (2H, d), 0.38 (2H, m), 0.93 (1H, m), 1.38 (9H, m), 3.08 (2H, s), 4.44 (2H, s), 7.22 (2H, s), 8.29 (1H, s).

LC/MS, t=3.37 min, [M−¹Bu⁺] 291.

### Intermediate 19: [2-(3 Chloro-4-fluoro-phenylamino)-4-trifluoromethyl-pyrimidin-5-ylmethyl]-cyclopropylmethyl-carbamic acid dimethyl-ethyl ester

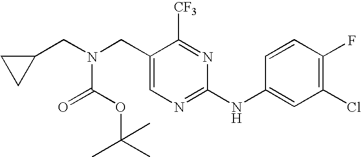

A mixture of (2-amino-4-trifluoromethyl-pyrimidin-5-ylmethyl)-cyclopropylmethyl-carbamic acid dimethyl-ethyl ester, (100 mg), 4-bromo-2-chloro-1-fluorobenzene (60 mg), cesium carbonate (131 mg), tris(dibenzylideneacetone)dipalladium (0) (3 mg) and 4,5-bis(diphenylphosphino)-9,9-dimethylxanthene (2 mg) and 1,4-dioxan (1 ml) was heated to 100° C. under nitrogen for 24 h. A mixture of tris(dibenzylideneacetone)dipalladium (0) (3 mg) and 4,5-bis(diphenylphosphino)-9,9-dimethylxanthene (2 mg) was added 3 times at two hour intervals, and the reaction allowed to cool. The mixture was diluted with ethyl acetate, washed with water, dried (MgSO₄), and evaporated under reduced pressure. The residue was purified using the MDAP system detailed at the beginning of the experimental section to afford the title compound as a yellow solid (63 mg).

NMR (DMSO-d6) δ 0.15 (2H, d), 0.40 (2H, m), 0.97 (1H, m), 1.32 (9H, m), 3.15 (2H, s), 4.55 (2H, s), 7.39 (1H, t), 7.67 (11H, m), 8.05 (1H, m), 8.58 (1H, s), 10.40 (1H, s).

LC/MS, t=4.50 min, molecular ion observed [M+H⁺] 475, consistent with C₂₁H₂₃F₄ClN₄O₂.

### Intermediate 20: 2-(2,4-Dichlorophenylamino)-4-trifluoromethylpyrimidine-5-carboxylic acid

(a). To a solution of methyl 2-chloro-4-trifluoromethylpyrimidine-5-carboxylate (0.5 g, ex Maybridge) in 1,4-dioxan (5 ml) was added 2,4-dichloroaniline (1.7 g) and the solution stirred at reflux temperature for 24 h. 1,4-Dioxan was removed under reduced pressure and ethyl acetate (15 ml) added. The solution was washed sequentially with 2N hydrochloric acid (10 ml) and water (3×10 ml), dried (MgSO₄), evaporated and triturated with hexane to afford methyl 2-(2,4-dichlorophenyl-amino)-4-trifluoromethyl-pyrimidine-5-carboxylate (214 mg).

NMR (400 MHz, CDCl₃) δ 3.95 (3H, s), 7.33 (1H, d), 7.46 (1H, d), 7.99 (1H, s), 8.48 (1H, d), 9.06 (11H, s).

LC/MS, t=3.74 min, [MH⁺] 366 and 368.

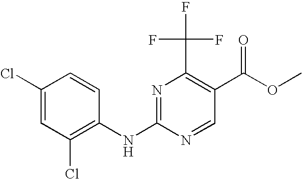

(b). To a solution of methyl 2-(2,4-dichlorophenylamino)-4-trifluoro-methylpyrimidine-5-carboxylate (0.21 g) in ethanol (15 ml) was added a solution of potassium hydroxide (205 mg) in ethanol (10 ml) and the solution stirred at reflux for 15 h. Ethanol was removed under reduced pressure and water (15 ml) added. The solution was washed with ether and concentrated hydrochloric acid added to adjust the acidity to pH 1. The precipitated solid was filtered, washed with water and dried in vacuo at 50° C. to afford 2-(2,4-dichlorophenylamino)-4-trifluoro-methylpyrimidine-5-carboxylic acid (0.18 g).

NMR (400 MHz, DMSO-d6) δ 7.47 (1H, d), 7.60 (1H, d), 7.75 (1H, s), 8.96 (1H, s), 10.3 (1H, s), 13.6 (1H, s).

LC/MS, t 4.17 min, [MH⁺—CO₂] 306 and 308.

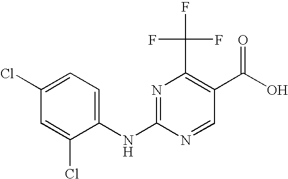

### Intermediate 21: [2-(2,4-Dichlorophenylamino)-4-trifluoromethyl-pyrimidin-5-yl]-methanol

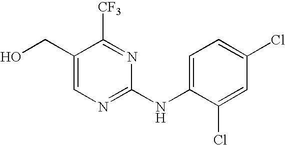

To a solution of 2-(2,4-dichlorophenylamino)-4-trifluoromethyl-pyrimidine-5-carboxylic acid (3.0 g), in 1,2-dimethoxyethane (53 ml) at −20° C. was added N-methylmorpholine (1.09 ml) followed by isobutyl chloroformate (1.38 ml). After 10 minutes, the precipitated N-ethylmorpholine hydrochloride was removed by filtration and the filtrate treated at −20° C. with a solution of sodium borohydride (540 mg) in water (15 ml). After a further 5 minutes, the reaction was quenched by the addition of water. The aqueous mixture was extracted with ethyl acetate (3×150 ml), and the combined, dried (MgSO₄) organic extracts were evaporated. The residue was purified by Biotage Horizon chromatography eluting with 5-50% ethyl acetate:isohexane to afford the title compound (0.69 g).

NMR (DMSO-d6) δ 4.54 (2H, d), 5.42 (1H, t), 7.44 (1H, dd), 7.64 (1H, d), 7.68 (1H, d), 8.71 (1H, s), 9.63 (1H, s).

LC/MS, t=3.57 min, molecular ion observed [MH⁺] 338, consistent with C₁₂H₈Cl₂F₃N₃O.

### Intermediate 22: 2-(2,4-Dichlorophenylamino)-4-trifluoromethyl-pyrimidine-5-carbaldehyde

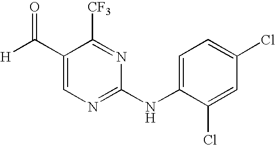

To a solution of [2-(2,4-dichlorophenylamino)-4-trifluoromethyl-pyrimidin-5-yl]-methanol, (0.69 g) in dichloromethane (25 ml) was added sodium chloride (1.67 g) and manganese (IV) oxide (1.77 g), and the mixture stirred at room temperature for 48 h. The mixture was filtered onto a bed of Celite washing with dichloromethane. The filtrate was evaporated under reduced pressure to afford the title compound (0.6 g).

NMR (DMSO-d6) δ 7.50 (1H, dd), 7.58 (1H, d), 7.75 (1H, d), 9.05 (1H, s), 10.00 (1H, s), 10.75 (1H, s).

LC/MS, t=3.62 min, molecular ion observed [MH⁺] 336, consistent with C₁₂H₆Cl₂F₃N₃O.

### Intermediate 23: 2-(3-fluorophenylamino)-4-trifluoromethylpyrimidine-5-carboxylic acid

(a). In a manner similar to Intermediate 9, methyl 2-chloro-4-trifluoromethylpyrimidine-5-carboxylate (0.5 g) and 3-fluoroaniline (1.16 g) afforded methyl 2-(3-fluorophenylamino)-4-trifluoromethyl-pyrimidine-5-carboxylate (0.65 g).

NMR (400 MHz, DMSO-d6) δ 3.88 (3H, s), 6.95 (1H, t of d), 7.40 (1H, q), 7.54 (1H, d), 7.79 (1H, d of t), 9.12 (1H, s), 10.95 (1H, s).

LC/MS, t=3.50 min, [MH⁺] 316.

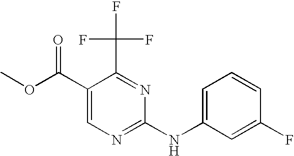

(b). In a manner similar to Intermediate 10, methyl 2-(3-fluorophenylamino)-4-trifluoro-methylpyrimidine-5-carboxylate (0.65 g) afforded 2-(3-fluorophenylamino)-4-trifluoromethylpyrimidine-5-carboxylic acid (0.54 g).

NMR (400 MHz, DMSO-d6) δ 6.90 (1H, t of d), 7.39 (1H, q), 7.55 (1H, d), 7.80 (1H, d of t), 9.10 (1H, s), 10.85 (1H, s), 13.7 (1H, br s)

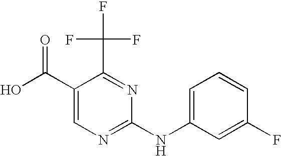

### Intermediate 24: [2-(3-Fluorophenylamino)-4-trifluoromethyl-pyrimidin-5-yl]-methanol

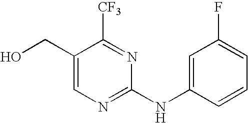

Prepared and purified in a similar manner to [2-(2,4-dichlorophenylamino)-4-trifluoromethyl-pyrimidin-5-yl]-methanol (Intermediate 21), using 2-(3-fluorophenylamino)-4-trifluoromethyl-pyrimidine-5-carboxylic acid to afford the title compound (1.98 g).

NMR (DMSO-d6) δ 4.57 (2H, d), 5.48 (1H, s), 6.85 (1H, m), 7.33 (1H, q), 7.50 (1H, dd), 7.79 (1H, dt), 8.85 (1H, s), 10.40 (1H, s).

LC/MS, t=3.20 min, molecular ion observed [MH⁺] 288, consistent with C₁₂H₉F₄N₃O

### Intermediate 25: 2-(3-Fluorophenylamino)-4-trifluoromethyl-pyrimidine-5-carbaldehyde

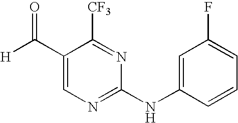

Prepared and purified in a similar manner to 7-(3-chlorophenylamino)-4-trifluoromethyl-pyrimidine-5-carbaldehyde (Intermediate 12), using [2-(3-fluorophenylamino)-4-trifluoromethyl-pyrimidin-5-yl]-methanol, to afford the title compound (1.78 g).

NMR (DMSO-d6) δ 6.97 (1H, m), 7.42 (1H, q), 7.56 (1H, dd), 7.79 (1H, dd), 9.18 (1H, s), 10.07 (1H, s), 11.17 (1H, s).

LC/MS, t=3.42 min, molecular ion observed [MH⁺] 286, consistent with C₁₂H₇F₄N₃O.

### Intermediate 26: 2-(3-Cyanophenylamino)-4-trifluoromethyl-pyrimidine-5-carboxylic acid methyl ester

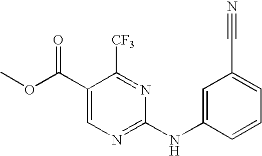

To a solution of methyl 2-chloro-4-trifluoromethyl-pyrimidine-5-carboxylate (4.0 g, ex Maybridge) in 1,4-dioxan (20 ml) was added 3-cyanoaniline (5.88 g) and the solution stirred under reflux for 3 h. 1,4-Dioxan was removed under reduced pressure and the residue stirred in 2N hydrochloric acid (100 ml) for 2 h. The solid was filtered onto a sinter, washed with 2N hydrochloric acid (2×50 ml), water (5×50 ml) and then sucked dry.

LC/MS, t=3.38 min, molecular ion observed [MH⁺] 323, consistent with C₁₄H₉F₃N₄O₂.

### Intermediate 27: 2-(3-Cyanophenylamino)-4-trifluoromethyl-pyrimidine-5-carboxylic acid

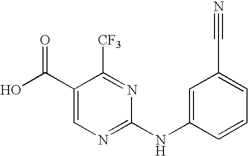

To a solution of 2-(3-cyanophenylamino)-4-trifluoromethyl-pyrimidine-5-carboxylic acid methyl ester, in tetrahydrofuran (40 ml) was added a solution of lithium hydroxide monohydrate (2.09 g) in water (13 ml) and the mixture stirred at room temperature overnight. The solvent was removed under reduced pressure and water added. The solution was washed with ether and concentrated hydrochloric acid was added to adjust the acidity to pH 1. The precipitate was filtered onto a sinter and washed with water until neutral to afford the title compound (4.68 g).

NMR (DMSO-d6) δ 7.56 (2H, m), 8.00 (1H, d), 8.27 (1H, s), 9.11 (1H, s), 10.95 (1H, s), 13.70 (1H, s).

LC/MS, t=3.32 min, molecular ion observed [MH⁺] 309, consistent with C₁₄H₉F₃N₄O₂.

### Intermediate 28: [2-(3-Cyanophenylamino)-4-trifluoromethyl-pyrimidin-5-yl]-methanol

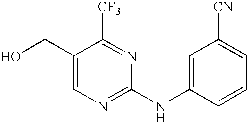

Prepared and purified in a similar manner to [2-(3-chlorophenylamino)-4-trifluoromethyl-pyrimidin-5-yl]-methanol (Intermediate 11), using 2-(3-cyanophenylamino)-4-trifluoromethyl-pyrimidine-5-carboxylic acid, to afford the title compound (2.41 g).

NMR (DMSO-d6) δ 4.58 (2H, d), 5.50 (1H, t), 7.45 (1H, d), 7.54 (1H, t), 7.99 (1H, dd), 8.01 (1H, s), 8.88 (1H, s), 10.40 (1H, s).

LC/MS, t=3.04 min, molecular ion observed [MH⁺] 295, consistent with C₁₃H₉F₃N₄O.

### Intermediate 29: 2-(3-Cyanophenylamino)-4-trifluoromethyl-pyrimidine-5-carbaldehyde

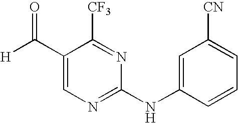

Prepared and purified in a similar manner to 2-(3-chlorophenylamino)-4-trifluoromethyl-pyrimidine-5-carbaldehyde (Intermediate 12), using [2-(3-cyanophenylamino)-4-trifluoromethyl-pyrimidin-5-yl]-methanol, to afford the title compound (2.22 g).

NMR (DMSO-d6) δ 7.61 (2H, m), 8.03 (1H, dd), 8.28 (1H, S), 9.20 (1H, s), 10.10 (1H, s), 11.30 (1H, s).

LC/MS, t=3.26 min, molecular ion observed [t] 293, consistent with C₁₃H₇F₃N₄O.

### Intermediate 30: 1-[2-(3-Chlorophenylamino)-4-trifluoromethyl-pyrimidin-5-yl]-ethanol

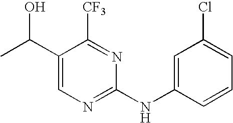

To a solution of 2-(3-chlorophenylamino)-4-trifluoromethyl-pyrimidine-5-carbaldehyde, (1.15 g) in tetrahydrofuran (25 ml) at −78° C. under nitrogen was added methylmagnesium bromide (3.0M in diethyl ether, 3.43 ml) dropwise. After 1 h, the reaction was quenched by the addition of saturated ammonium chloride (50 ml). and allowed to warm to room temperature. The aqueous was extracted with dichloromethane (2×40 ml) and the combined dried (Na₂SO₄) organic extracts were evaporated in vacuo. The residue was purified by Biotage flash chromatography over Merck 9385 silica gel eluting with 4:1 isohexane:ethyl acetate to afford the title compound (1.1 g).

NMR (DMSO) δ 1.40 (3H, d), 5.00 (1H, t), 5.60 (1H, d), 7.05 (1H, dd), 7.33 (1H, t), 7.66 (1H, dd), 7.97 (1H, t), 9.00 (1H, s), 10.35 (1H, s).

LC/MS, t=3.59 min, molecular ion observed [MH⁺] 318, consistent with C₁₃H₁₁ClF₃N₃O.

### Intermediate 31: 1-[2-(3-Chlorophenylamino)-4-trifluoromethyl-pyrimidin-5-yl]-ethanone

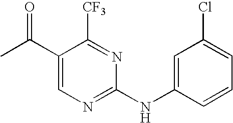

To a solution of 1-[2-(3-chlorophenylamino)-4-trifluoromethyl-pyrimidin-5-yl]-ethanol, (1.1 g) in dichloromethane (30 ml) was added sodium chloride (2.83 g) and manganese (IV) oxide (3.0 g), and the mixture stirred at room temperature for 72 h. The mixture was then heated under reflux overnight. The mixture was filtered onto a bed of Celite washing with dichloromethane. The filtrate was evaporated under reduced pressure to afford the title compound (520 mg).

NMR (DMSO) δ 2.60 (3H, s), 7.13 (1H, dd), 7.38 (1H, t), 7.70 (1H, dd), 8.03 (1H, t), 9.50 (1H, s), 10.85 (1H, s).

LC/MS, t=3.62 min, [MH⁺] 316.

### Intermediate 32: 1-[2-(3-Fluorophenylamino)-4-trifluoromethyl-pyrimidin-5-yl]-ethanol

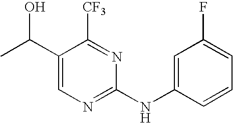

Prepared and purified in a similar manner to 1-[2-(3-chlorophenylamino)-4-trifluoromethyl-pyrimidin-5-yl]-ethanol (Intermediate 30), using 2-(3-fluorophenylamino)-4-trifluoromethyl-pyrimidine-5-carbaldehyde, to afford the title compound (0.99 g).

NMR (DMSO) δ 1.40 (3H, d), 5.00 (1H, t), 5.60 (1H, d), 6.80 (1H, m), 7.35 (1H, t), 7.50 (1H, dd), 7.80 (1H, dt), 9.00 (1H, s), 10.40 (1H, s). LC/MS, t=3.37 min, molecular ion observed [MH⁺] 302, consistent with C₁₃H₁₁F₄N₃O.

### Intermediate-33: 1-[2-(3-Fluorophenylamino)trifluoromethyl-pyrimidin-5-yl]-ethanone

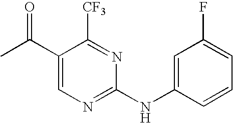

Prepared and purified in a similar manner to 1-[2-(3-chlorophenylamino)-4-trifluoromethyl-pyrimidin-5-yl]-ethanone (Intermediate 31), using 1-[2-(3-fluorophenylamino)-4-trifluoromethyl-pyrimidin-5-yl]-ethanol, to afford the title compound (0.58 g).

NMR (DMSO) δ 2.60 (3H, s), 7.20 (1H, m), 7.38 (1H, q), 7.50 (1H, dd), 7.80 (1H, dt), 9.25 (1H, s), 10.85 (1H, s).

LC/MS, t=3.42 min, molecular ion observed [MH⁺] 300, consistent with C₁₃H₉F₄N₃O.

### Intermediate 34: 2-(2,4-Dichlorophenylamino)-4-trifluoromethyl-pyrimidine-5-carboxylic acid methoxy-methyl-amide

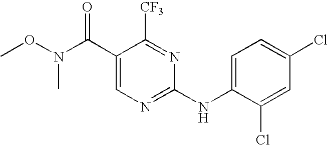

To a solution of 2-(2,4-dichlorophenylamino)-4-trifluoromethyl-pyrimidine-5-carboxylic acid, (3.0 g) in dimethylformamide (30 ml) was added N-ethyl morpholine (2.94 g), 1-hydroxybenzotriazole hydrate (1.799 g), 1-(3-dimethylamino-propyl)-3-ethylcarbodiimide hydrochloride (2.94 g) and N,O-dimethylhydroxylamine hydrochloride (0.99 g). The solution was stirred for 3 h and allowed to stand overnight. Dimethylformamide was removed under reduced pressure and ethyl acetate (50 ml) added. The solution was washed sequentially with 5% sodium bicarbonate solution (50 ml), water (50 ml), 5% citric acid solution (50 ml) and brine (50 ml), dried (MgSO₄) and evaporated to afford the title compound (3.13 g).

NMR (DMSO) δ 3.25 (3H, s), 3.49 (3H, s), 7.46 (1H, d), 7.61 (1H, d), 7.72 (1H, d), 8.76 (1H, s), 10.10 (1H, s).

LC/MS, t=3.50 min, molecular ion observed [MH⁺] 395, consistent with C₁₄H₁₁Cl₂F₃N₄O.

### Intermediate 35: 1-[2-(2,4-Dichlorophenylamino)-4-trifluoromethyl-pyrimidin-5-yl]-ethanone

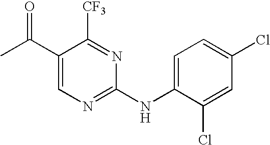

To a solution of 2-(2,4-dichlorophenylamino)-4-trifluoromethyl-pyrimidine-5-carboxylic acid methoxy-methyl-amide, (0.5 g), in dry tetrahydrofuran (10 ml) at −10° C. under nitrogen was added dropwise methyl lithium (1.6M in diethyl ether, 1.58 ml) and the solution stirred at 0° C. for 3 h. Further methyl lithium (1.6M in diethyl ether, 1.58 ml) was added at −10° C. dropwise and after 10 minutes the reaction was quenched by the addition of aqueous saturated ammonium chloride. The reaction was allowed to warm to room temperature, and the aqueous was extracted with ethyl acetate. The combined dried (Na₂SO₄) organic extracts were evaporated, and the residue purified by Biotage flash chromatography over Merck 9385 silica gel eluting with 7:3 isohexane:ethyl acetate to afford the title compound (0.11 g).

NMR (DMSO) δ 2.56 (3H, s), 7.57 (1H, dd), 7.73 (1H, d), 7.75 (1H, d), 9.07 (1H, s), 10.35 (1H, s).

LC/MS, t=3.63 min, molecular ion observed [MH⁺] 350, consistent with C₁₃H₈Cl₂F₃N₃O.

### Intermediate 36: C-(2-Fluoro-pyridin-4-yl)-methylamine dihydrochloride

### (a). 4-Bromomethyl-2-fluoro-pyridine

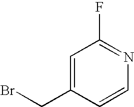

To a solution of 2-fluoro-4-methylpyridine (1.0 g, ex Lancaster) in carbon tetrachloride (10 ml) was added N-bromosuccinimide (1.6 g, ex Lancaster) and 1,1′-azobis (cyclohexanecarbonitrile) (100 mg, ex Aldrich). The mixture was then refluxed for 24 h. Carbon tetrachloride was removed under reduced pressure and the crude oily solid was used in the next stage without purification.

LC/MS, t=2.38 min, [MH⁺] 190 consistent with C₆H₅⁷⁹BrFN.

### (b). (2-Fluoro-pyridin-4-ylmethyl)-carbamic acid tert-butyl ester

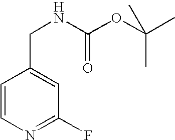

To crude 4-bromomethyl-2-fluoro-pyridine in an ice bath was added 25% ammonia solution (10 ml, ex BDH) and the mixture stirred at 0° for 5 h. Ammonia solution was removed under reduced pressure and the yellow oily solid residue dissolved in dichloromethane (10 ml) and dimethylformamide (1 ml). The solution was cooled in an ice bath and triethylamine (1.5 ml, ex BDH) was added followed by di-tert-butyl dicarbonate (1.0 g, ex Avocado). The solution was stirred at 0° for 1 h and then the dichloromethane removed under reduced pressure. The residue was dissolved in ethyl acetate and washed twice with water, dried (MgSO₄) and evaporated to give a yellow oil. This was purified by Biotage chromatography (100 g, silica column) eluting with 30% ethyl acetate in hexane to afford the title compound as a white solid (358 mg).

NMR (400 MHz, DMSO-d6) δ 1.40 (9K, s), 4.20 (2H, d), 6.97 (1H, s), 7.20 (1H, d), 7.60 (1H, t), 8.17 (1H, d)

LC/MS, t=2.60 min, [M−Me2C═CH2+H]⁺ 171, consistent with C₁₁H₁₅FN₂O₂

### (c). C-(2-Fluoro-pyridin-4-yl)-methylamine dihydrochloride

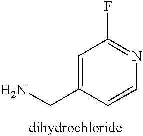

(2-Fluoro-pyridin-4-ylmethyl)-carbamic acid tert-butyl ester (350 mg) was treated at room temperature with 4N hydrochloric acid in 1,4-dioxan (5 ml) and stirred for 2 h. The white precipitate was filtered, washed with fresh ether and dried to afford the title compound (200 mg).

NMR (400 MHz, DMSO-d6) δ 4.14 (2H, d), 7.38 (1H, s), 7.51 (1H, d), 8.28 (1H, d), 8.82 (3H, s).

### Intermediate 37: 2-Dimethylaminomethylene-4,4-dimethyl-3-oxo-pentanoic acid ethyl ester

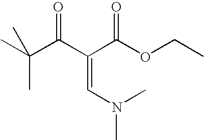

Ethyl pivaloylacetate (Ex Avacado) (99.6 g) and N,N-dimethylformamide dimethyl acetal (172 g) were heated with stirring at 100° C. for 4 hours. The reaction mixture was allowed to cool and the volatiles removed in vacuo. The residue was washed with aqueous 1M NaOH and the organic layer washed with brine, dried (MgSO₄), and the volatiles were removed in vacuo to yield the title compound as an orange oil (131 g).

NMR (d⁶-DMSO) δ 0.95-1.20 (12H, m), 2.83 (6H, brs), 3.95-4.10 (2H, m), 7.30 (1H, s).

### Intermediate 38: Ethyl 4-(1,1-dimethylethyl)-2-hydroxy-5-pyrimidinecarboxylate

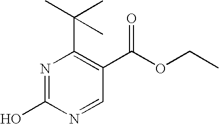

2-Dimethylaminomethylene-4,4-dimethyl-3-oxo-pentanoic acid ethyl ester (71.5 g) and urea (20.79 g) were heated at 155° C. in glacial acetic acid (300 ml) for 16 hours. The volatiles were removed in vacuo and the residue co-evaporated twice with toluene. The residue was triturated with iso-hexane and filtered off to yield the title compound as a yellow crystalline solid (17.2 g).

NMR (d⁶-DMSO) δ 1.23-1.35 (12H, m), 4.22 (2H, q), 8.23 (1H, s), 12.20 (1H, brs).

LC/MS t=2.15 min [MH⁺]=225 consistent with molecular formula C, H₁₆N₂O₃

### Intermediate 39: Ethyl 2-chloro-4-(1,1-dimethylethyl)-5-pyrimidinecarboxylate

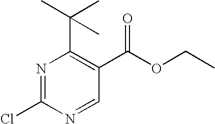

Ethyl 4-(1,1-dimethylethyl)-2-hydroxy-5-pyrimidinecarboxylate (15.56 g) was suspended in phenyldichlorophosphate (150 ml) and was stirred at 180° C. for 2 hours. The reaction mixture was poured onto ice (excess) and the mixture was basified to pH 7 using solid NaHCO₃. The reaction mixture was dissolved in EtOAc and washed with water. The organic layer was washed with brine, dried (MgSO₄) and the volatiles were removed in vacuo to yield the title compound as a brown liquid (12.5 g).

NMR (d⁶-DMSO) δ 1.26-1.40 (12H, m), 4.37 (2H, q), 8.85 (1H, s).

LC/MS t=3.26 min [MH⁺]=243 consistent with molecular formula C₁₁H₁₅³⁵ClN₂O₂

### Intermediate 40: 4-tert-Butyl-2-(3-chloro-phenylamino)-pyrimidine-5-carboxylic acid ethyl ester

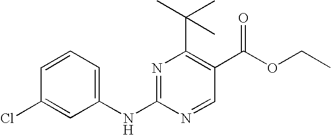

3-Chloroaniline (4.00 ml) was added to a solution of ethyl 2-chloro-4-(1,1-dimethylethyl)-5-pyrimidinecarboxylate (3.00 g) in dioxan (10 ml) and the solution stirred at 100° C. for 3 hours. The volatiles were removed in vacuo and the residue dissolved in EtOAc and washed three times with aqueous 2M HCl. The organic layer was then washed with brine, dried (MgSO₄), and the volatiles were removed in vacuo to yield the title compound as a brown oil which solidified (3.99 g).

NMR (d⁶-DMSO) δ 1.32 (3H, t), 1.39 (9H, s), 4.30 (2H, q), 7.05 (1H, d), 7.33 (1H, t), 7.61 (1H, d), 8.01 (1H, s), 8.60 (1H, s).

LC/MS t=4.22 min [MH⁺]=334 consistent with molecular formula C₁₇H₂₀³⁵ClN₃O₂

### Intermediate 41: 4-tert-Butyl-2-(3-chloro-phenylamino)-pyrimidine-5-carboxylic acid

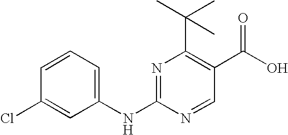

The title compound was prepared from 4-tert-butyl-2-(3-chloro-phenylamino)-pyrimidine-5-carboxylic acid ethyl ester (3.79 g) in a manner similar to the preparation of intermediate 20, but with a reflux time of 6.5 hours, to yield the title compound as an off white solid (3.02 g).

NMR (d⁶-DMSO) δ 1.42 (9H, s), 7.04 (1H, d), 7.32 (1H, t), 7.62 (1H, d), 8.10 (1H, s), 8.61 (1H, s).

LC/MS t=4.10 min [MH⁺]=306 consistent with molecular formula C₁₅H₁₆³⁵ClN₃O₂

### Intermediate 42: [4-tert-Butyl-2-(3-chloro-phenylamino)-pyrimidin-5-yl]-methanol

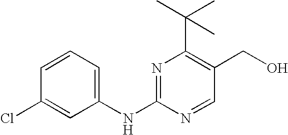

The title compound was prepared from 4-tert-butyl-2-(3-chloro-phenylamino)-pyrimidine-5-carboxylic acid (2.09 g) in a manner similar to intermediate 3, except that 5 minutes after the addition of the NaBH₄ (390 mg) in water (3 mL), a further addition of sodium borohydride (390 mg) in water (3 mL) was made. After 5 minutes the reaction was quenched with water and worked up as before to yield the title compound as a pale yellow solid (1.63 g).

NMR (d⁶-DMSO) δ 1.40 (9H, s), 4.63 (2H, d), 5.18 (1H, t), 6.95 (1H, d), 7.28 (1H, t), 7.59 (1H, d), 8.12 (1H, s), 8.42 (1H, s), 9.74 (1H, s).

LC/MS t=3.60 min [MH⁺]=292 consistent with molecular formula C₁₅H₁₈³⁵ClN₃O

### Intermediate 43: 4-tert-Butyl-2-(3-chloro-phenylamino)-pyrimidine-5-carbaldehyde

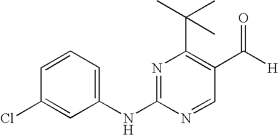

The title compound was prepared from [4-tert-butyl-2-(3-chloro-phenylamino)pyrimidin-5-yl]-methanol (340 mg) in a manner similar to intermediate 4 except that the reaction was heated for 5 hours, then stirred overnight, before being worked up in the same way to yield the title compound as a pale yellow solid (276 mg).

NMR (d⁶-DMSO) δ 1.46 (9H, s), 7.10 (1H, d), 7.36 (1H, t), 7.66 (1H, d), 8.17 (1H, s), 8.87 (1H, s), 10.33 (1H, s), 10.50 (1H brs).

LC/MS [MH⁺]=290 consistent with molecular formula C₁₅H₁₆³⁵ClN₃H

### EXAMPLE 1

### (3-Chloro-phenyl)-(5-cyclobutylaminomethyl-4-cyclopropyl-pyrimidin-2-yl)-amine

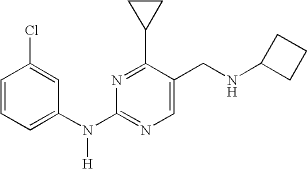

2-(3-Chloro-phenylamino)-4-cyclopropyl-pyrimidine-5-carbaldehyde, (0.150 g, 0.0548 mmol, 1 eq) in methanol (2 ml) was added to 4A molecular sieves. Aminocyclobutane (0.150 g, 0.219 mmol, 4 eq) was added, and the tube shaken for 1 hour. Glacial acetic acid (0.23 g, 0.384 mmol, 7 eq) in dichloromethane (0.75 ml) and MP-cyanoborohydride polymer reagent (Part number 800406, Argonaut Technologies Inc, 0.540 g, 0.109 mmol, 2 eq) were added and the reaction mixture shaken for 48 hours. The polymer was filtered off, washed with methanol, and the filtrate evaporated in vacuo. The residue was purified by chromatography using the Biotage Horizon system described at the beginning of the experimental, to yield the title compound (0.034 g, 0.0104 mmol, 19%) as a white solid.

¹H NMR (CDCl₃) 1.37-1.50 (4H, m), 1.75-2.08 (2H, m), 2.32 (3H, brs), 2.56-2.69 (2H, m), 3.93 (1H, brs), 4.42 (2H, brs), 7.12 (1H, dd, J=8 Hz, J=1 Hz), 7.23 (1H, t, J=8 Hz), 7.39 (1H, d, J=8 Hz), 7.65 (1H, s), 9.41 (1H, s), 10.23 (1H, s), 11.05 (1H, s).

LC/MS t=2.49 min. Molecular ion observed [MH⁺] consistent with molecular formula C₁₈H₂₁³⁵ClN₄

### EXAMPLE 2

### (3-Chloro-phenyl)-{5-[(cyclopropylmethyl-amino)-methyl]-4-isopropyl-pyrimidin-2-yl}-amine

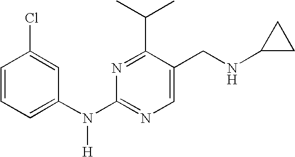

The title compound prepared from 2-(3-chloro-phenylamino)-4-isopropyl-pyrimidine-5-carbaldehyde, and cyclopropylamine using the procedure described for Example 1 to yield the title compound.

NMR (DMSO-d6) δ −0.16-0.02 (2H, m), 0.011-0.17 (2H, m), 1.00 (6H, d, J=7 Hz), 1.80-1.87 (1H, m) 3.04-3.16 (1H, m), 3.46 (2H, s), 6.71 (1H, dd, J=8 Hz, 2 Hz), 7.04 (1H, t, J=8 Hz), 7.43 (1H, dd, J=8 Hz, J=1 Hz), 7.95 (1H, t, J=2 Hz), 8.08 (1H, s), 9.45 (1H, s).

LC/MS t=2.51 min. Molecular ion observed for [MH⁺] consistent with molecular formula C₁₇H₂₁³⁵ClN₄.

The compounds in Table 1 below were all prepared in a similar manner to the preparation of Example 1 by reductive amination of the appropriate aldehyde 2-(3-chloro-phenylamino)-4-cyclopropyl-pyrimidine-5-carbaldehyde or 2-(3-chloro-phenylamino)-4-isopropyl-pyrimidine-5-carbaldehyde with the appropriate amine. The amines used in the reductive aminations were all commercially available except for cyclopentylmethylamine hydrochloride which was prepared as described by Kelley et al in J Med Chem, 1997, 40, 3207, and used in place of the free base. The purification methods are given in the appropriate column of the Table:

Purification method A: Biotage 25S silica gel cartridge, using DCM/MeOH/AcOH/water-120:15:3:2 as elutant
Purification method B: Trituration with MeOH
Purification method C: Biotage Horizon used, conditions given earlier.
Purification method D: MDAP
Purification method E: Biotage 25+M silica gel cartridge, using EtOAc:Isohexane 50:50 to 70:30, then trituration with MeOH
Purification method F: Biotage 25+M silica gel cartridge, EtOAc:isohexane 50:50. To product in Et₂O add cHCl, & evaporate
Purification method G: Biotage 25+M silica gel cartridge, EtOAc:isohexane 50:50 to 100% EtOAc.

To product in Et₂O add cHCl, and evaporate

TABLE 1

| Example | Name | Structure |
|---|---|---|
| 3 | (3-Chloro-phenyl)-{4-cyclopropyl-5-[(cyclopropylmethyl-amino)-methyl]-pyrimidin-2-yl}-amineacetate | 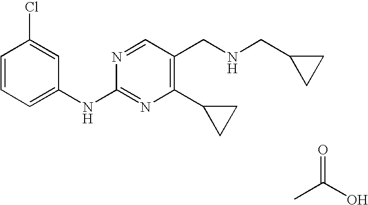 |
| 4 | (2-Chloro-phenyl)-(4-cyclopropyl-5-morpholin-4-ylmethyl-pyrimidin-2-yl)-amine | 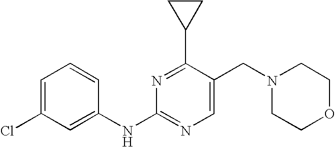 |
| 5 | (3-Chloro-phenyl)-[4-cyclopropyl-5-(isobutylamino-methyl)-pyrimidin-2-yl]-amine | 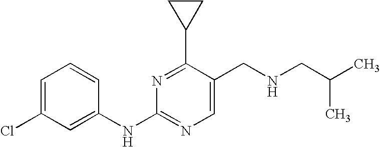 |
| 6 | (3-Chloro-phenyl)-(4-isopropyl-5-morpholin-4-ylmethyl-pyrimidin-2-yl)-amine | 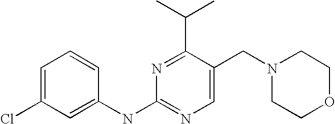 |
| 7 | (3-Chloro-phenyl)-{5-[(cyclopropylmethyl-amino)-methyl]-4-isopropyl-pyrimidin-2-yl}-amine hydrochloride | 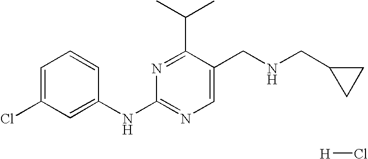 |
| 8 | (3-Chloro-phenyl)-{5-[(cyclopentylmethyl-amino)-methyl]-4-isopropyl-pyrimidin-2-yl}-amine hydrochloride | 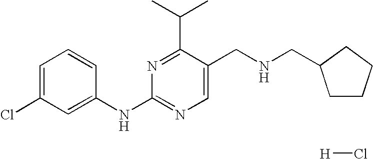 |
| 9 | (3-Chloro-phenyl)-{4-isopropyl-5-[(2-methoxy-ethylamino)-methyl]-pyrimidin-2-yl}-amineformate | 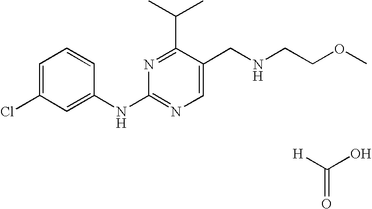 |

| | | | LC/MS |
|---|---|---|---|
| | | | Retention Time, |
| | | | t (min) [M+] |
| | | | Molecular formula |
| | Prep | Purification | of free base |
| Example | Method | Method | NMR data |
| 3 | A | A | LC/MS t = 2.46. |
| | | | NMR (CDCl₃) δ 0.26 (2 H, d, J = 5 Hz), 0.59 (2 H, d, |
| | | | J = 5 Hz), 1.01-1.33 (5 H, m), 2.11-2.20 (1 H, m), 2.73 |
| | | | (2 H, d, J = 7 Hz), 4.06 (2 H, s) 6.97 (1 H, d, J = 7 Hz), 7.19 |
| | | | (1 H, t, J = 8 Hz), 7.33 (1 H, d, J = 8 Hz), 7.87 (1 H, s), 8.18 |
| | | | (1 H, s), 8.67 (1 H, brs), 9.80 (1 H, brs). |
| 4 | A | B | LC/MS t = 2.57. |
| | | | NMR (DMSO) δ 1.03-1.12 (4 H, m), 2.36-2.44 (4 H, |
| | | | m), 2.64-2.68 (1 H, m), 3.49 (2 H, s) 3.53-3.59 (4 H, m), |
| | | | 6.94 (1 H, dd, J = 8 Hz, J = 2 Hz), 7.27 (1 H, t, J = 8 Hz), 7.56 |
| | | | (1 H, dd, J = 8 Hz, J = 1 Hz), 8.01 (1 H, t, J = 2 Hz), 8.18 |
| | | | (1 H, s), 9.64 (1 H, s). |
| 5 | A | C | LC/MS t = 2.42 |
| | | | [MH+] 331. |
| | | | Consistent with molecular formula C₁₈H₂₃³⁵ClN₄ |
| 6 | B | E | LC/MS t = 2.79. |
| | | | NMR (DMSO-d6) δ 1.23 (6 H, d, J = 6 Hz), 2.35 (4 H, |
| | | | brs), 3.32-3.42 (1 H, m), 3.54 (4 H, t, J = 4 Hz), 6.94 (1 H, |
| | | | dd, J = 8 Hz, J = 1 Hz), 7.19 (1 H, t, J = 8 Hz), 7.33 (1 H, d, |
| | | | J = 8 Hz), 7.28 (1 H, t, J = 8 Hz), 7.64 (1 H, dd, J = 8 Hz, |
| | | | J = 1 Hz), 8.14 (1 H, t, J = 2 Hz), 8.25 (1 H, s), 9.73 (1 H, s). |
| 7 | B | F | LC/MS t = 2.56. |
| | | | NMR (DMSO-d6) δ 0.39-0.45 (2 H, m), 0.56-0.63 (2 H, |
| | | | m), 1.14-1.30 (7 H, m), 2.87-2.96 (2 H, m), 3.32-3.43 |
| | | | (1 H, m) 4.13 (2 H, t, J = 8 Hz), 6.43 (>2 H, brs), 7.00 (1 H, |
| | | | dd, J = 8 Hz, J = 2 Hz), 7.32 (1 H, t, J = 8 Hz), 7.66 (1 H, dd, |
| | | | J = 8 Hz, J = 1 Hz), 8.15 (1 H, t, J = 1 Hz), 8.67 (1 H, s), 9.64 |
| | | | (1 H, brs), 10.00, (1 H, s). |
| 8 | B | G | LC/MS t = 2.91. |
| | | | NMR (DMSO-d6) δ 0.89-1.30 (10 H, m), 1.57-1.87 |
| | | | (5 H, m), 2.85 (2 H, brs), 3.36 (1 H, t, J = 8 Hz), 4.12 (2 H, |
| | | | brs), 5.37 (>2 H, brs), 6.99 (1 H, d, J = 8 Hz), 7.32 (1 H, t, |
| | | | J = 8 Hz), 7.65 (1 H, d, J = 8 Hz), 8.14 (1 H, s), 8.63 (1 H, |
| | | | s), 9.02 (1 H, brs), 9.93 (1 H, s). |
| 9 | A | D | LC/MS t = 2.44 |
| | | | [MH+] 335. |
| | | | Consistent with molecular formula C₁₇H₂₃³⁵ClN₄O |

### EXAMPLE 10

### (3-Chlorophenyl){5-[(cyclopentylmethylamino)-methyl]-4 trifluoromethyl-pyrimidin-2-yl}-amine, formate

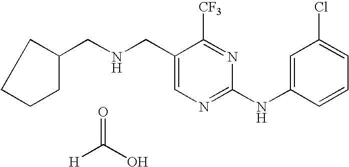

To a solution of 2-(3-chlorophenylamino)-4-trifluoromethyl-pyrimidine-5-carbaldehyde, (120 mg) in methanol (2 ml) was added powdered 4A molecular sieves (100 mg) followed by cyclopentylmethylamine hydrochloride (106 mg prepared as described in Kelley et al., J. Med. Chem., 40, 3207, (1997)) in methanol (2 ml). The mixture was shaken in a capped Alltech extract-clean filter column (8 ml) for 2 h. Glacial acetic acid (136 uL) was added followed by MP-Cyanoborohydride (Argonaut Technologies) (390 mg) and the mixture shaken for 6 h. The mixture was filtered and the MP-Cyanoborohydride washed with methanol (2×4 ml). The filtrate was applied to a methanol conditioned SCX column (2 g) and eluted with methanol. The column was then eluted with 2% 880 ammonia in methanol and the solution collected and evaporated under reduced pressure. Purified by Mass-directed autopurification using the procedures detailed at the beginning of the experimental to afford (3-chlorophenyl)-{5-[(cyclopentylmethylamino)-methyl]-4-trifluoromethyl-pyrimidin-2-yl}-amine, formate (64 mg).

NMR (MeOD) δ 1.28 (2H, m), 1.69 (4H, m), 1.90 (2H, m), 2.19 (1H, m), 2.99 (2H, d), 4.20 (2H, s), 7.04 (1H, dd), 7.29 (1H, t), 7.57 (1H, dd), 7.97 (1H, s), 8.43 (1H, s), 8.80 (1H, s).

LC/MS, t=2.76 min, molecular ion observed [MH⁺] 385, consistent with C₁₈H₂₀ClF₃N₄

### EXAMPLE 11

### (3-Chlorophenyl){5-[(isobutylamino)-methyl]-4-trifluoromethyl-pyrimidin-2yl}-amine, formate

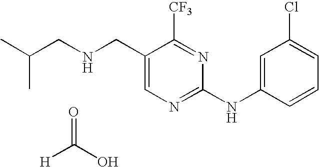

To a solution of 2-(3-chlorophenylamino)-4-trifluoromethyl-pyrimidine-5-carbaldehyde, (60 mg) in methanol (1 ml) was added powdered 4A molecular sieves (30 mg) followed by isobutylamine (15 mg) in methanol (1 ml). The mixture was shaken in a capped Alltech extract-clean filter column (8 ml) for 1 h. Polymer-supported borohydride on Amberlite (Aldrich) (95 mg) was added and the mixture shaken overnight. The mixture was filtered and the polymer-supported borohydride washed with methanol (2×4 ml). The combined filtrate was applied to a methanol conditioned SCX column (2 g) and eluted with methanol. The column was then eluted with 2% aqueous 0.880 ammonia in methanol and the eluant evaporated under reduced pressure. The residue was purified by MDAP using the procedures detailed at the beginning of the experimental to afford (3-chlorophenyl)-{5-[(isobutylamino)-methyl]-4-trifluoromethyl-pyrimidin-2yl}-amine, formate (30 mg).

NMR (MeOD) δ 1.00 (6H, d), 2.00 (1H, m), 1.90 (2H, m), 2.85 (2H, d), 4.20 (2H, s), 7.04 (1H, dd), 7.29 (1H, t), 7.57 (1H, dd), 7.97 (1H, s), 8.43 (1H, s), 8.80 (1H, s).

LC/MS, t=2.78 min, molecular ion observed [M−H⁺] 357.

### EXAMPLE 12

### (3-Chlorophenyl)-{[5-(cyclohexylmethylamino)-methyl]-4-trifluoromethyl-pyrimidin-2yl}-amine

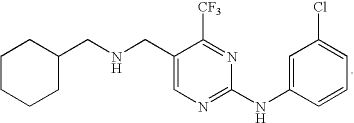

To a solution of (5-bromomethyl-4-trifluoromethyl-pyrimidin-2-yl)-(3-chloro-phenyl)-amine, (25 mg) in tetrahydrofuran (1 ml) was added a solution of cyclohexanemethylamine (200 mg) in tetrahydrofuran (1 ml) and the solution stirred at room temperature for 30 min. The solvent was removed under reduced pressure. The residue was co-evaporated from dichloromethane and a few drops of triethylamine. The residue was purified by chromatography on a Waters Se-pak cartridge of silica gel, eluting with ether: dichloromethane 1:10 to afford the title compound as a white solid (24 mg).

NMR (DMSO-d6) δ 0.85 (2H, q), 1.14-1.23 (>3H, m), 1.4 (1H, m), 1.63-1.75 (5H, m), 2.34 (2H, d), 3.73 (2H, s), 7.03 (1H, dd), 7.33 (1H, t), 7.66 (1H, dd), 7.99 (1H, s), 8.86 (1H, s), 10.30 (1H, s).

LC/MS, t=2.92 min, molecular ion observed [MH⁺] 399, consistent with C₁₉H₂₂³⁵ClF₃N₄

### EXAMPLE 13

### A (3-Chloro-4-fluoro-phenyl)-{5-[(cyclopropylmethyl-amino)-methyl]-4-trifluoromethyl-pyrimidin-2-yl}-amine hydrochloride

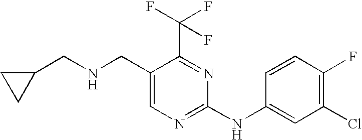

To [2-(3-chloro-4-fluoro-phenylamino)-4-trifluoromethyl-pyrimidin-5-ylmethyl]-cyclopropylmethyl-carbamic acid dimethyl-ethyl ester, (58 mg) was added 4N hydrogen chloride in dioxan (2 ml) and the solution stirred at room temperature for 1 h. The solution was evaporated under reduced pressure to afford the title compound as a yellow foam (50 mg).

NMR (DMSO-d6) δ 0.39 (2H, m), 0.59 (2H, m), 1.12 (1H, m), 2.91 (2H, d), 4.22 (2H, s), 7.42 (11H, t), 7.68 (1H, m), 8.06 (11H, m), 9.06 (1H, s), 9.39 (2H, s), 10.40 (1H, s).

LC/MS, t=2.65 min, molecular ion observed [M+H⁺] 375, consistent with C₁₆H₁₅ClF₄N₄.

The Examples in the Table 2 were prepared in a similar manner to the methods described above:

Prep Method D: Reductive amination as described for Example 96 using four equivalents of the appropriate amine.
Prep Method E: Reductive amination as described for method D using the appropriate known amine and aldehyde, the syntheses of which are described above.
Prep Method F: Reductive amination as described for method E using two to four equivalents of the appropriate amine and tetrahydrofuran as the solvent.
Prep Method G: Treatment of the corresponding BOC compound with 4N hydrogen chloride in 1,4-dioxan.
Prep Method H: As described for Example 12
Purification method A: Purify using an SCX column followed by the MDAP system detailed at the beginning of the experimental section as described for Example 10
Purification method B: Purify using the MDAP system detailed at the beginning of the experimental section.
Purification method C: Purify using an SCX column followed by the Biotage Horizon system detailed at the beginning of the experimental section.
Purification method D: Purify using the Biotage Horizon system detailed at the beginning of the experimental section.
Purification method E: Purify by trituration with methanol.
Purification method F: Purify by trituration with diethyl ether.
Purification method G: Purify as described for Example 12.

Example 26 was prepared using tetrahydro-2H-thiopyran-4-amine 1,1-dioxide hydrochloride which may be prepared as described by N Sakai in Patent No WO 2003072554.

TABLE 2

| | | | | | Ret Time |
|---|---|---|---|---|---|
| | | | | Puri- | (min) [M+] |
| Ex- | | | Prep | fica- | Molecular |
| am- | | | Me- | Me- | formula of |
| ple | Name | Structure | thod | thod | free base |
| 14 | (3-Chlorophenyl)-(5-morpholin-4-ylmethyl-4-trifluoromethyl-pyrimidin-2-yl)-amine. | 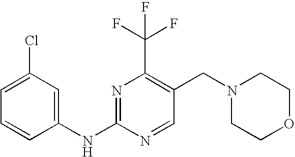 | F | A | 3.19373C₁₆H₁₆³⁵ClF₃N₄O |
| 15 | (3-Chlorophenyl)-(5-{[(tetrahydro-pyran-4-ylmethyl)-amino]-methyl}-4-trifluoromethyl-pyrimidin-2-yl)-amine,formate | 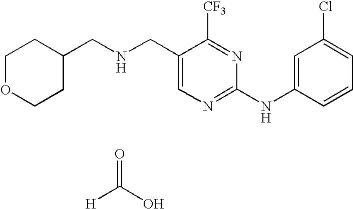 | D | A | 2.52401C₁₈H₂₀³⁵ClF₃N₄O |
| 16 | (3-Chlorophenyl)-{5-[(tetrahydro-pyran-4-ylamino)-methyl]-4-trifluoromethyl-pyrimidin-2-yl}-amine, formate | 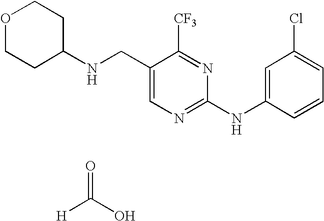 | D | A | 2.49387C₁₇H₁₈³⁵ClF₃N₄O |
| 17 | (3-Chlorophenyl)-{5-[(2-methoxy-ethylamino)-methyl]-4-trifluoromethyl-pyrimidin-2-yl}-amine, formate | 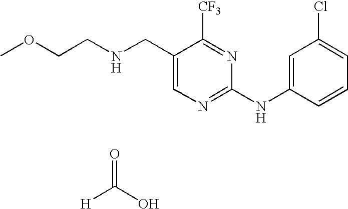 | D | A | 2.51361C₁₅H₁₆³⁵ClF₃N₄O |
| 18 | (3-Chlorophenyl)-{5-[(cyclobutylmethylamino)-methyl]-4-trifluoromethyl-pyrimidin-2-yl}-amine,formate | 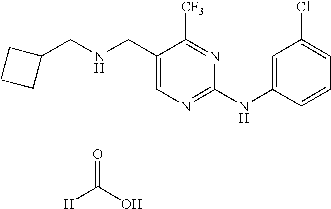 | D | A | 2.70371C₁₇H₁₈³⁵ClF₃N₄ |
| 19 | (3-Chlorophenyl)-(5-propylaminomethyl-4-trifluoromethyl-pyrimidin-2-yl)-amine,formate | 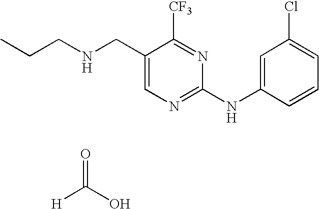 | D | A | 2.56345C₁₅H₁₆³⁵ClF₃N₄ |
| 20 | (3-Chlorophenyl)-{5-[(2,2-dimethylpropylamino)-methyl]-4-trifluoromethyl-pyrimidin-2-yl}-amine. | 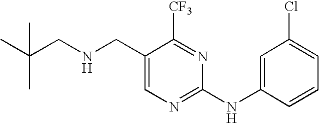 | D | D | 2.74373C₁₇H₂₀³⁵ClF₃N₄ |
| 21 | (3-Chlorophenyl)-(5-cyclobutylaminomethyl-4-trifluoromethyl-pyrimidin-2-yl)-amine. | 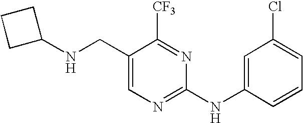 | D | D | 2.59357C₁₆H₁₆³⁵ClF₃N₄ |
| 22 | (3-Chlorophenyl)-(5-butylaminomethyl-4-trifluoromethyl-pyrimidin-2-yl)-amine,formate | 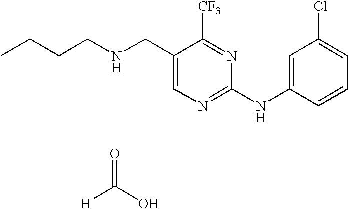 | D | A | 2.67359C₁₆H₁₈³⁵ClF₃N₄ |
| 23 | (3-Chlorophenyl)-(5-cyclopropylaminomethyl-4-trifluoromethyl-pyrimidin-2-yl)-amine,formate | 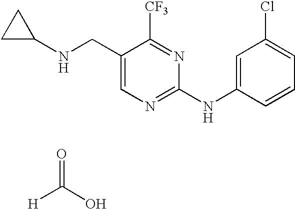 | D | A | 2.61343C₁₅H₁₄³⁵ClF₃N₄ |
| 24 | (3-Chlorophenyl)-{5-[(3-methylbutylamino)-methyl]-4-trifluoromethyl-pyrimidin-2-yl}-amine,formate. | 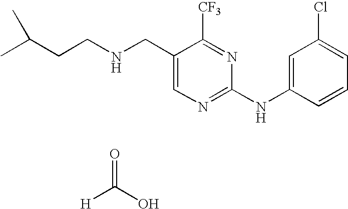 | D | A | 2.78373C₁₇H₂₀³⁵ClF₃N₄ |
| 25 | (3-Chlorophenyl)-{5-[(cyclopropylmethylamino)-methyl]-4-trifluoromethyl-pyrimidin-2-yl}-amine,formate. | 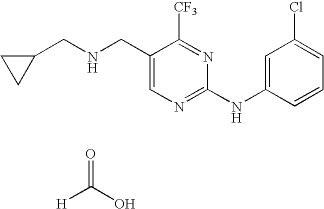 | D | A | 2.60357C₁₆H₁₆³⁵ClF₃N₄ |
| 26 | (3-Chlorophenyl)-{5-[(1,1-dioxo-hexahydro-1l⁶-thiopyran-4-ylamino)-methyl]-4-trifluoromethyl-pyrimidin-2-yl}-amine, formate. | 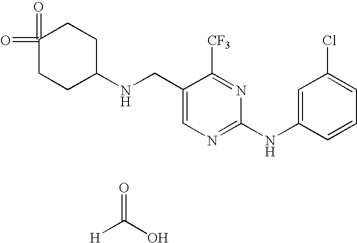 | D | B | 2.74432C₁₇H₁₈³⁵ClF₃N₄O₂S |
| 27 | (2,4-Dichlorophenyl)-{5-[(isobutylamino)-methyl]-4-trifluoromethyl-pyrimidin-2yl}-amine. | 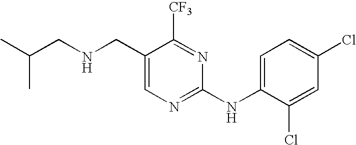 | E | C | 2.71393C₁₆H₁₇³⁵Cl₂F₃N₄ |
| 28 | (2,4-Dichlorophenyl)-{5-[(cyclopropylmethylamino)-methyl]-4-trifluoromethyl-pyrimidin-2-yl}-amine. | 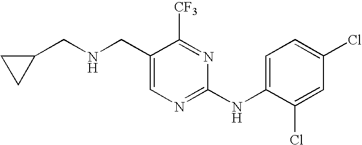 | E | C | 2.67391C₁₆H₁₅³⁵Cl₂F₃N₄ |
| 29 | (2,4-Dichlorophenyl)-{5-[(cyclobutylmethylamino)-methyl]-4-trifluoromethyl-pyrimidin-2-yl}-amine,formate. | 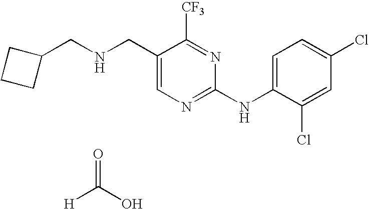 | E | A | 2.80405C₁₇H₁₇³⁵Cl₂F₃N₄ |
| 30 | (2,4-Dichlorophenyl)-(5-cyclopropylaminomethyl-4-trifluoromethyl-pyrimidin-2-yl)-amine. | 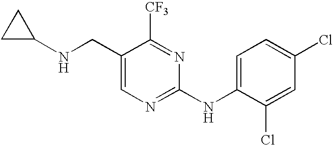 | E | A | 2.74377C₁₅H₁₃³⁵Cl₂F₃N₄ |
| 31 | (3-Chlorophenyl)-(5-{[(pyridin-3-ylmethyl)-amino]-methyl}-4-trifluoromethyl-pyrimidin-2-yl}-amine,formate. | 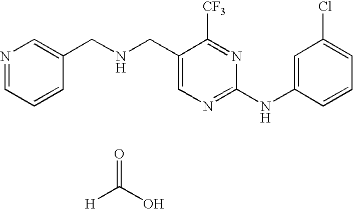 | D | B | 2.65394C₁₈H₁₅³⁵ClF₃N₅ |
| 32 | (2,4-Dichlorophenyl)-{5-[(2,2-dimethylpropylamino)-methyl]-4-trifluoromethyl-pyrimidin-2-yl}-amine. | 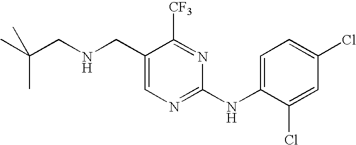 | E | B | 2.86407C₁₇H₁₉³⁵Cl₂F₃N₄ |
| 33 | (2,4-Dichlorophenyl)-{5-[(2-methoxy-ethylamino)-methyl]-4-trifluoromethyl-pyrimidin-2-yl}-amine. | 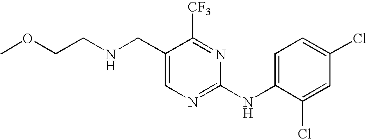 | E | B | 2.60395C₁₅H₁₅³⁵Cl₂F₃N₄O |
| 34 | (3-Chlorophenyl-(5-cyclopentylaminomethyl-4-trifluoromethyl-pyrimidin-2-yl)-amine. | 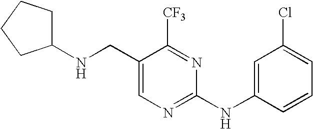 | D | D | 2.66371C₁₇H₁₈³⁵ClF₃N₄ |
| 35 | (2,4-Dichlorophenyl)-(5-morpholin-4-ylmethyl-4-trifluoromethyl-pyrimidin-2-yl)-amine. | 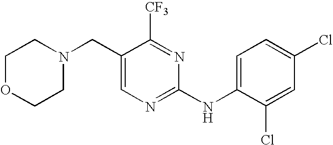 | F | E | 3.54407C₁₆H₁₅³⁵Cl₂F₃N₄O |
| 36 | (3-Fluorophenyl)-{5-[(isobutylamino)-methyl]-4-trifluoromethyl-pyrimidin-2yl}-amine, formate | 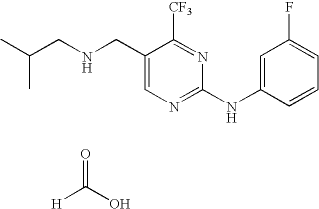 | E | B | 2.55343C₁₆H₁₈F₄N₄ |
| 37 | (3-Fluorophenyl)-{5-[(cyclobutylmethylamino)-methyl]-4-trifluoromethyl-pyrimidin-2-yl}-amine,formate | 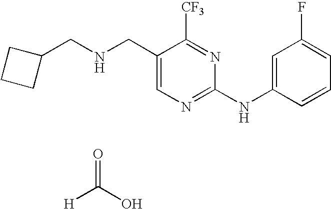 | E | B | 2.63355C₁₇H₁₈F₄N₄ |
| 38 | (3-Fluorophenyl)-(5-cyclopropylaminomethyl-4-trifluoromethyl-pyrimidin-2-yl)-amine. | 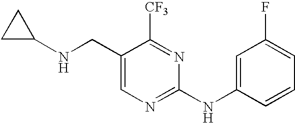 | E | B | 2.46327C₁₅H₁₄F₄N₄ |
| 39 | (3-Chlorophenyl)-(5-piperidin-1-ylmethyl-4-trifluoromethyl-pyrimidin-2-yl)-amine,formate | 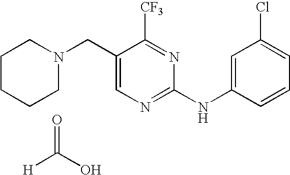 | F | A | 2.60371C₁₇H₁₈³⁵ClF₃N₄ |
| 40 | (3-Chlorophenyl)-(5-pyrrolidin-1-ylmethyl-4-trifluoromethyl-pyrimidin-2-yl)-amine, formate. | 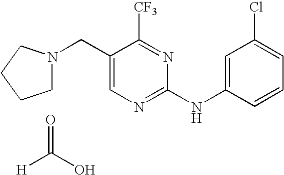 | F | A | 2.52357C₁₆H₁₆³⁵ClF₃N₄ |
| 41 | (3-Chlorophenyl)-{5-[(4-fluorobenzylamino)-methyl]-4-trifluoromethyl-pyrimidin-2-yl)-amine,formate | 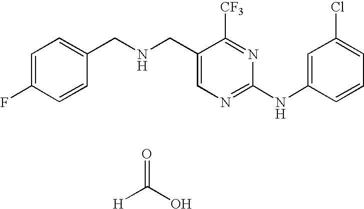 | D | A | 2.89411C₁₉H₁₅³⁵ClF₄N₄ |
| 42 | (3-Chlorophenyl)-(5-{[methyl-(tetrahydro-pyran-4-ylmethyl)-amino]-methyl}-4-trifluoromethyl-pyrimidin-2-yl)-amine | 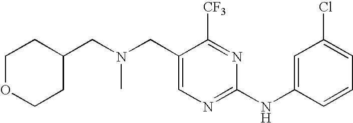 | F | A | 2.60415C₁₉H₂₂³⁵ClF₃N₄O |
| 43 | (3-Chlorophenyl)-(5-cyclohexylaminomethyl-4-trifluoromethyl-pyrimidin-2-yl)-amine. | 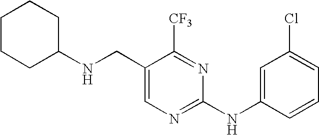 | D | C | 2.72385C₁₈H₂₀³⁵ClF₃N₄ |
| 44 | (3-Chlorophenyl)-(5-dimethylaminomethyl-4-trifluoromethyl-pyrimidin-2-yl)-amine, formate. | 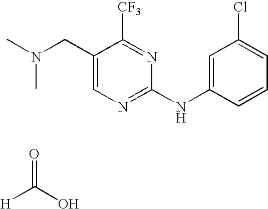 | D | A | 2.46331C₁₄H₁₄³⁵ClF₃N₄ |
| 45 | (3-Chlorophenyl)-[5-(phenethylamino-methyl)-4-trifluoromethyl-pyrimidin-2-yl]-amine, formate. | 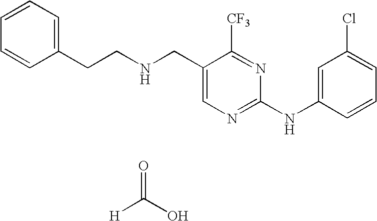 | D | B | 2.83407C₂₀H₁₈³⁵ClF₃N₄ |
| 46 | (3-Chlorophenyl)-[5-(phenpropylamino-methyl)-4-trifluoromethyl-pyrimidin-2-yl]-amine, formate. | 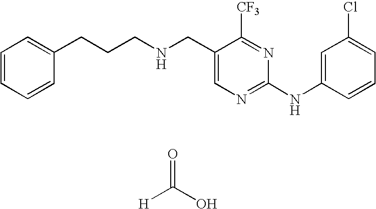 | D | B | 2.92421C₂₁H₂₀³⁵ClF₃N₄ |
| 47 | {5-[(Butyl-methyl-amino)-methyl]-4-trifluoromethyl-pyrimidin-2-yl}-(3-chlorophenyl)-amine | 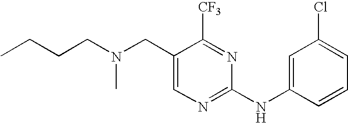 | D | A | 2.73373C₁₇H₂₀³⁵ClF₃N₄ |
| 48 | [5-(sec-Butylamino-methyl)-4-trifluoromethyl-pyrimidin-2-yl]-(3-chloro-phenyl)-amine, formate. | 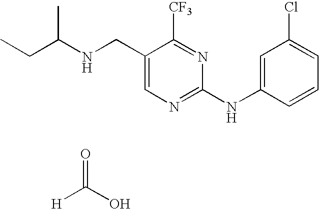 | D | A | 2.63359C₁₆H₁₈³⁵ClF₃N₄ |
| 49 | (3-Chlorophenyl)-(5-{[[(2R)-tetrahydro-furan-2-ylmethyl]-amino]-methyl}-4-trifluoromethyl-pyrimidin-2-yl)-amine,formate. | 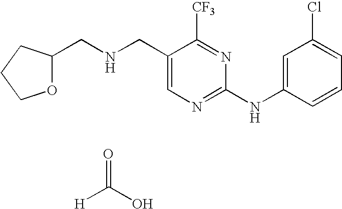 | D | A | 2.62387C₁₇H₁₈³⁵ClF₃N₄O |
| 50 | (3-Chlorophenyl)-(5-{[[(2R)-tetrahydro-furan-2-ylmethyl]-amino]-methyl}-4-trifluoromethyl-pyrimidin-2-yl)-amine,formate. | 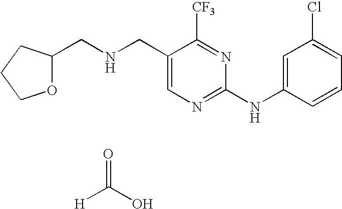 | D | A | 2.61387C₁₇H₁₈³⁵ClF₃N₄O |
| 51 | 3-Chlorophenyl)-{5-[(cyclobutylmethyl-methyl-amino)-methyl]-4-trifluoromethyl-pyrimidin-2-yl}-amine | 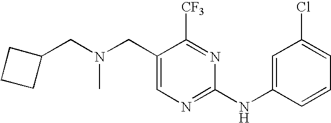 | F | D | 2.78385C₁₈H₂₀³⁵ClF₃N₄ |
| 52 | 1-{[2-(3-Chlorophenylamino)-4-trifluoromethyl-pyrimidin-5-ylmethyl]-amino}-propan-2-ol | 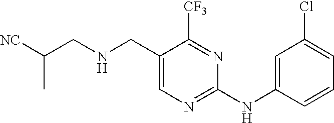 | D | D | 2.46361C₁₅H₁₆³⁵ClF₃N₄O |
| 53 | (2,4-Dichlorophenyl)-(5-pyrrolidin-1-ylmethyl-4-trifluoromethyl-pyrimidin-2-yl)-amine. | 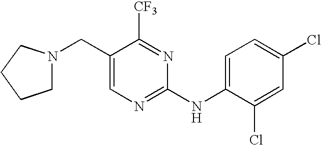 | F | D | 2.69405C₁₇H₁₇³⁵Cl₂F₃N₄ |
| 54 | (3-Fluorophenyl)-{5-[(cyclopropylmethylamino)-methyl]-4-trifluoromethyl-pyrimidin-2-yl}-amine,formate | 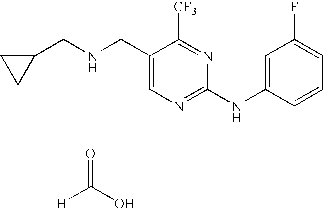 | E | B | 2.51341C₁₆H₁₆F₄N₄ |
| 55 | (3-Fluorophenyl)-{5-[(3-methylbutylamino)-methyl]-4-trifluoromethyl-pyrimidin-2-yl}-amine. | 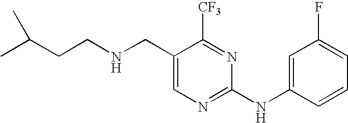 | E | D | 2.69357C₁₇H₂₀F₄N₄ |
| 56 | (3-Fluorophenyl)-(5-morpholin-4-ylmethyl-4-trifluoromethyl-pyrimidin-2-yl)-amine. | 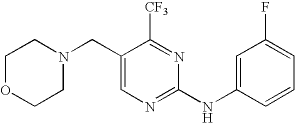 | F | B | 2.98357C₁₆H₁₆F₄N₄O |
| 57 | (3-Chlorophenyl)-{5-[(2,2-dimethylpropylamino)-methyl]-4-trifluoromethyl-pyrimidin-2-yl}-amine,formate. | 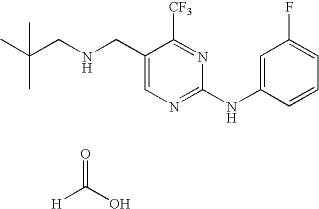 | E | B | 2.67357C₁₇H₂₀F₄N₄ |
| 58 | (3-Cyanophenyl)-(5-cyclopropylaminomethyl-4-trifluoromethyl-pyrimidin-2-yl)-amine,formate | 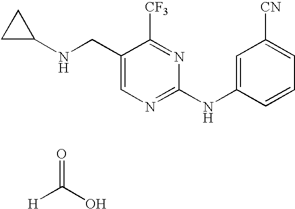 | E | B | 2.36334C₁₆H₁₄F₃N₅ |
| 59 | (3-Cyanophenyl)-{5-[(isobutylamino)-methyl]-4-trifluoromethyl-pyrimidin-2yl}-amine, formate | 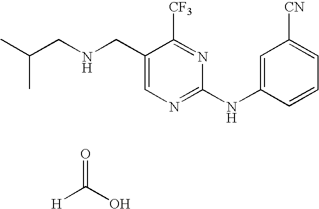 | E | B | 2.45350C₁₇H₁₈F₃N₅ |
| 60 | (3-Chlorophenyl)-{5-[(cyclohexylmethyl-methyl-amino)-methyl]-4-trifluoromethyl-pyrimidin-2-yl}-amine, formate | 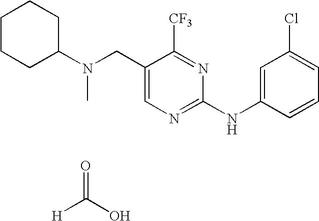 | F | A | 2.78399C₁₉H₂₂³⁵ClF₃N₄ |
| 61 | (3-Chlorophenyl)-{5-[(benzylmethyl-methyl-amino)-methyl]-4-trifluoromethyl-pyrimidin-2-yl}-amine, formate | 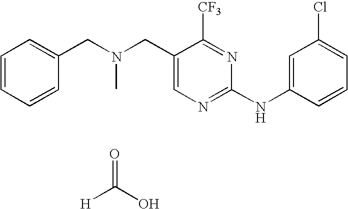 | F | A | 3.20407C₂₀H₁₈³⁵ClF₃N₄ |
| 62 | (3-Chlorophenyl)-(5-dipropylaminomethyl-4-trifluoromethyl-pyrimidin-2-yl)-amine. | 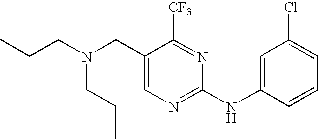 | D | B | 2.92387C₁₈H₂₂³⁵ClF₃N₄ |
| 63 | (3-Chlorophenyl)-[5-(isopropylamino-methyl)-4-trifluoromethyl-pyrimidin-2-yl]-amine. | 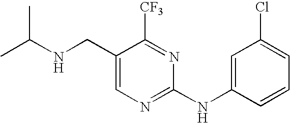 | D | D | 2.55345C₁₅H₁₆³⁵ClF₃N₄ |
| 64 | (3-Chloro-phenyl)-{5-[(1-methyl-piperidin-4-ylamino)-methyl]-4-trifluoromethyl-pyrimidin-2-yl}-amine,formate | 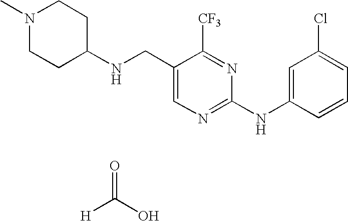 | D | B | 2.26400C₁₈H₂₁³⁵ClF₃N₅ |
| 65 | 4-({[2-(3-Chlorophenylamino)-4-trifluoromethyl-pyrimidin-5-ylmethyl]-amino}-methyl)-piperidine-1-carboxylicacid tert-butyl ester. | 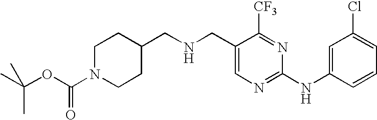 | D | D | 2.93500C₂₃H₂₉³⁵ClF₃N₅O₂ |
| 66 | (3-Chlorophenyl)-(5-{[(piperidin-4-ylmethyl)-amino]-methyl}-4-trifluoromethyl-pyrimidin-2-yl)-amine,dihydrochloride. | 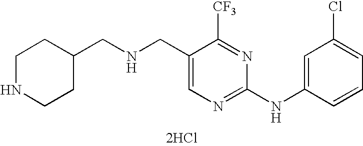 | G | F | 2.20400C₁₈H₂₁³⁵ClF₃N₅ |
| 67 | (3-Chlorophenyl)-{5-[(2-ethyl-butylamino)-methyl]-4-trifluoromethyl-pyrimidin-2-yl}-amine, formate | 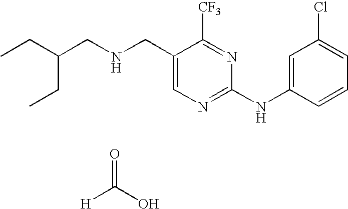 | D | B | 2.90387C₁₈H₂₂³⁵ClF₃N₄ |
| 68 | N-[2-(3-Chlorophenylamino)-4-trifluoromethyl-pyrimidin-5-ylmethyl]-N′,N′-dimethyl-ethane-1,2-diamine, formate | 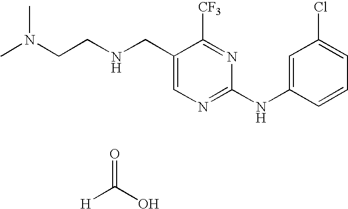 | D | B | 2.57374C₁₆H₁₉³⁵ClF₃N₅ |
| 69 | N-[2-(3-Chlorophenylamino)-4-trifluoromethyl-pyrimidin-5-ylmethyl]-N′,N′-dimethyl-propane-1,2-diamine, formate. | 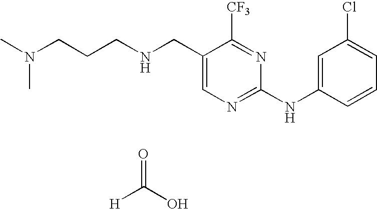 | D | A | 2.12388C₁₇H₂₁³⁵ClF₃N₅ |
| 70 | (3-Chlorophenyl)-(5-{[(pyridin-2-ylmethyl)-amino]-methyl}-4-trifluoromethyl-pyrimidin-2-yl)-amine. | 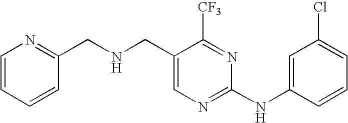 | D | B | 2.69394C₁₈H₁₅³⁵ClF₃N₅ |
| 71 | (3-Chlorophenyl)-{5-[(1-ethyl-propylamino)-methyl]-4-trifluoromethyl-pyrimidin-2-yl}-amine. | 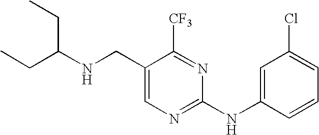 | D | D | 2.72373C₁₇H₂₀³⁵ClF₃N₄ |
| 72 | (3-Chlorophenyl)-{5-[(3,3-dimethyl-butylamino)-methyl]-4-trifluoromethyl-pyrimidin-2-yl}-amine. | 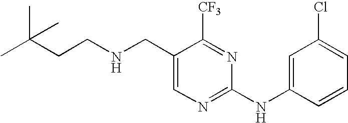 | D | D | 2.85387C₁₈H₂₂³⁵ClF₃N₄ |
| 73 | 1-({[2-(3-Chloro-phenylamino)-4-trifluoromethyl-pyrimidin-5-ylmethyl]-amino}-methyl)-cyclohexanol, formate | 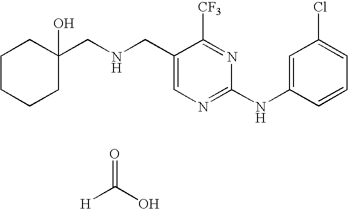 | D | B | 2.70415C₁₉H₂₂³⁵ClF₃N₄O |
| 74 | 2-{[2-(3-Chloro-phenylamino)-4-trifluoromethyl-pyrimidin-5-ylmethyl]-amino}-ethanol, formate. | 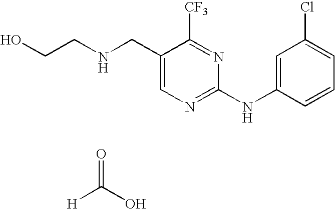 | D | B | 2.42347C₁₄H₁₄³⁵ClF₃N₄O |
| 75 | N-(2-{[2-(3-Chloro-phenylamino)-4-trifluoromethyl-pyrimidin-5-ylmethyl]-amino}-ethyl)-acetamide, formate | 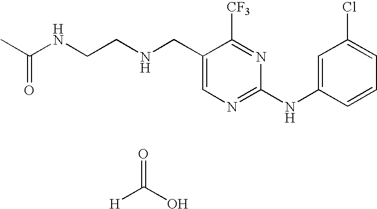 | D | B | 2.44388C₁₆H₁₇³⁵ClF₃N₅O |
| 76 | [5-(sec-Butylamino-methyl)-4-trifluoromethyl-pyrimidin-2-yl]-(3-fluoro-phenyl)-amine, formate. | 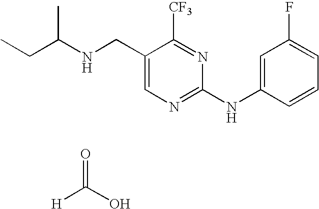 | E | B | 2.53343C₁₆H₁₈F₄N₄ |
| 77 | (3-Cyanophenyl)-(5-morpholin-4-ylmethyl-4-trifluoromethyl-pyrimidin-2-yl)-amine, formate. | 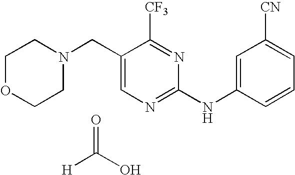 | E | A | 2.85364C₁₇H₁₆F₃N₅O |
| 78 | (3-Cyanophenyl)-{5-[(cyclopropylmethylamino)-methyl]-4-trifluoromethyl-pyrimidin-2-yl}-amine,formate. | 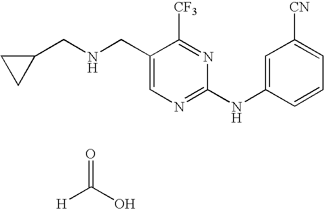 | E | B | 2.39348C₁₇H₁₆F₃N₅ |
| 79 | (3-Chloro-phenyl)-(5-{[(6-methoxy-pyridin-3-ylmethyl)-amino]-methyl}-4-trifluoromethyl-pyrimidin-2-yl}-amine,formate. | 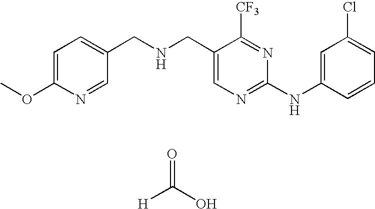 | D | B | 2.72424C₁₉H₁₇³⁵ClF₃N₅O |
| 80 | (5-Aminomethyl-4-trifluoromethyl-pyrimidin-2-yl)-(3-chlorophenyl)-amine. | 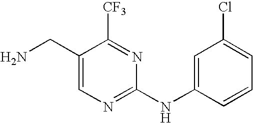 | H | G | 2.47303C₁₂H₁₀³⁵ClF₃N₄ |
| 81 | (3-Chlorophenyl)-[5-(4-methyl-piperazin-1-ylmethyl)-4-trifluoromethyl-pyrimidin-2-yl]-amine, formate. | 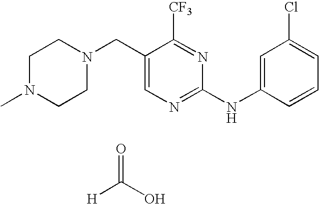 | F | A | 2.61386C₁₇H₁₉³⁵ClF₃N₅ |
| 82 | (3-Chlorophenyl)-(5-{[1-(4-fluoro-phenyl)-ethylamino]-methyl}-4-trifluoromethyl-pyrimidin-2-yl)-amine | 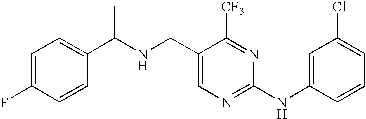 | D | D | 3.00425C₂₀H₁₇³⁵ClF₄N₄ |
| 83 | (5-{[Bis-(2-methoxy-ethyl)-amino]-methyl}-4-trifluoromethyl-pyrimidin-2-yl)-(3-chlorophenyl)-amine. | 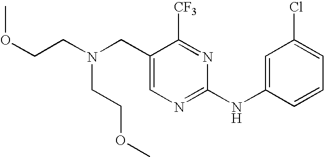 | D | D | 3.26419C₁₈H₂₂³⁵ClF₃N₄O₂ |
| 84 | {1-[2-(3-Chloro-phenylamino)-4-trifluoromethyl-pyrimidin-5-ylmethyl]-piperidin-4-yl}-methanol, formate. | 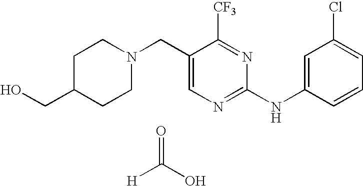 | F | B | 2.48401C₁₈H₂₀³⁵ClF₃N₄O |

### EXAMPLE 85

### 3-Chlorophenyl)-[5-(1-cyclopropylamino-ethyl)-4-trifluoromethyl-pyrimidin-2-yl]-amine

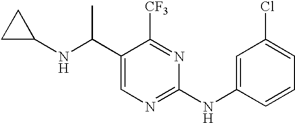

To a solution of 1-[2-(3-chlorophenylamino)-4-trifluoromethyl-pyrimidin-5-yl]-ethanone (80 mg) in methanol (1.0 ml) was added powdered 4A molecular sieves (70 mg) followed by cyclopropylamine (57 mg) in methanol (1.0 ml). The mixture was shaken in a capped Alltech extract-clean filter column (8 ml) for 0.5 h. Glacial acetic acid (106 mg) in dichloromethane (1 ml) was added followed by MP-Cyanoborohydride (Argonaut Technologies) (248 mg) and the mixture shaken for 84 h. MP-Cyanoborohydride (248 mg) was added and the mixture shaken for 20 h. The mixture was filtered and the MP-Cyanoborohydride washed with methanol (2×4 ml). The combined filtrates were evaporated under reduced pressure and the residue purified by Mass-directed autopurification using the procedures detailed at the beginning of the experimental to afford (3-chlorophenyl)[S-(1-cyclopropylamino-ethyl)-4-trifluoromethyl-pyrimidin-2-yl]-amine (29 mg).

NMR (MeOD) δ 0.21-0.45 (4H, m), 1.40 (3H, d), 1.95 (1H, m), 4.23 (1H, m), 6.95 (1H, d), 7.25 (1H, t), 7.58 (1H, d), 8.00 (1H, s), 9.50 (1H, s).

LC/MS, t=2.75 min, molecular ion observed [M−H⁺] 355, consistent with C₁₆H₁₆ClF₃N₄.

The Examples in Table 3 were prepared as follows.

Prep Method A: Reductive amination as described for Example 85
Prep Method B: Reductive amination as described for Example 85 using an excess of zinc chloride in tetrahydrofuran, and shaking overnight, prior to the addition of acetic acid and MP-cyanoborohydride.
Prep Method C: Reductive amination as described for Example 85 using titanium isopropoxide (2 eq) and microwave heating at 160° C. for 3×10 minutes to form the imine prior to the addition of the acetic acid and MP-Cyanoborohydride. Reaction times between 3 and 14 days.
Prep Method D: Reductive amination as described for Example 85 using zinc chloride and microwave heating at 180° C. for 15 minutes to form the imine prior to the addition of the acetic acid and MP-Cyanoborohydride.
Prep Method E: Reductive amination as described for Example 85 using tetrahydrofuran as solvent.
Purification method A: Purify using an SCX column followed by the MDAP system detailed at the beginning of the experimental section as described for Example 10.
Purification method B: Purify using the MDAP system detailed at the beginning of the experimental section.

TABLE 3

| | | | Prep- | | Ret Time |
|---|---|---|---|---|---|
| | | | ara- | Puri- | (min) [M+] |
| | | | tion | fica- | Molecular |
| | | | Me- | Me- | Formula of |
| Example | Name | Structure | thod | thod | free base |
| 86 | (3-Fluorophenyl)-[5-(1-cyclopropylamino-ethyl)-4-trifluoromethyl-pyrimidin-2-yl]-amine | 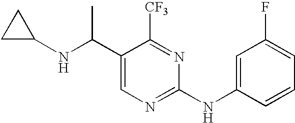 | B | B | 2.58341C₁₆H₁₆F₄N₄ |
| 87 | (3-Fluorophenyl)-[5-(1-isobutylamino-ethyl)-4-trifluoromethyl-pyrimidin-2-yl]-amine | 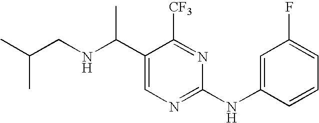 | B | B | 2.62357C₁₇H₂₀F₄N₄ |
| 88 | (3-Chlorophenyl)-(5-{1-[(tetrahydro-pyran-4-ylmethyl)-amino]-ethyl}-4-trifluoromethyl-pyrimidin-2-yl)-amine, formate | 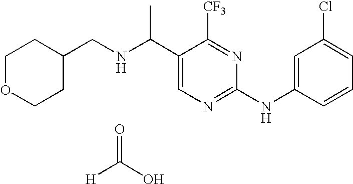 | C | B | 2.53415C₁₉H₂₂³⁵ClF₃N₄O |
| 89 | (3-Chlorophenyl)-{5-[1-(cyclohexylmethyl-amino)-ethyl]-4-trifluoromethyl-pyrimidin-2-yl}-amine, formate. | 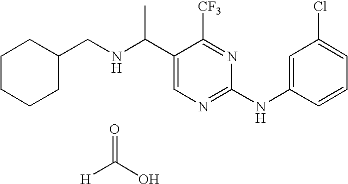 | C | A | 2.92413C₂₀H₂₄³⁵ClF₃N₄ |
| 90 | (3-Chlorophenyl)-{5-[1-(cyclopentylmethyl-amino)-ethyl]-4-trifluoromethyl-pyrimidin-2-yl}-amine, formate. | 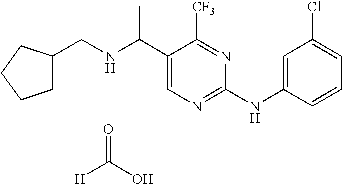 | C | A | 2.80399C₁₉H₂₂³⁵ClF₃N₄ |
| 91 | (2,4-Dichlorophenyl)-(5-{1-[(tetrahydro-pyran-4-ylmethyl)-amino]-ethyl}-4-trifluoromethyl-pyrimidin-2-yl)-amine. | 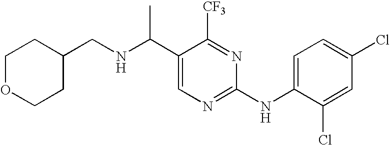 | C | B | 2.53449C₁₉H₂₁³⁵Cl₂F₃N₄O |
| 92 | (3-Chlorophenyl)-[5-(1-isobutylamino-ethyl)-4-trifluoromethyl-pyrimidin-2-yl]-amine, formate | 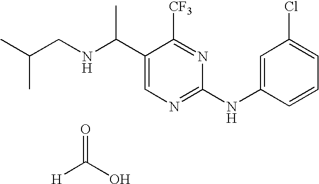 | D | B | 2.76373C₁₇H₂₀³⁵ClF₃N₄ |
| 93 | (3-Chlorophenyl)-[5-(1-morpholin-4-yl-ethyl)-4-trifluoromethyl-pyrimidin-2-yl]-amine | 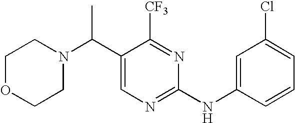 | E | B | 3.57387C₁₇H₁₈³⁵ClF₃N₄O |
| 94 | (3-Fluorophenyl)-{5-[1-(cyclopropylmethyl-amino)-ethyl]-4-trifluoromethyl-pyrimidin-2-yl}-amine, formate | 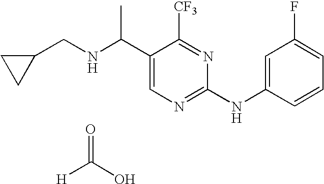 | A | B | 2.58355C₁₇H₂₈F₄N₄ |
| 95 | (2,4-Dichlorophenyl)-{5-[1-(cyclohexyhnethyl-amino)-ethyl]-4-trifluoromethyl-pyrimidin-2-yl}-amine. | 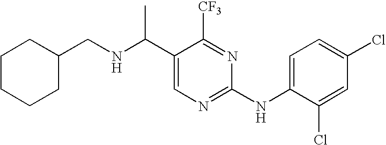 | C | B | 3.00447C₂₀H₂₃³⁵Cl₂F₃N₄ |

### EXAMPLE 96

### (3-Chlorophenyl)-(5-{[(2-fluoropyridin-4-ylmethyl)amino]-methyl}-4-trifluoromethyl-pyrimidin-2yl}-amine, formate

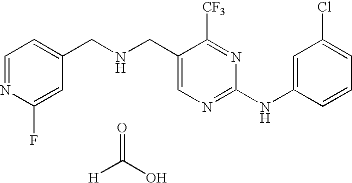

To a solution of 2-(3-chlorophenylamino)-4-trifluoromethyl-pyrimidine-5-carbaldehyde (100 mg) in methanol (1.5 ml) was added powdered 4A molecular sieves (70 mg) followed by C-(2-fluoro-pyridinyl)-methylamine dihydrochloride (Intermediate 36) (81 mg) in methanol (1.5 ml). The mixture was shaken in a capped Alltech extract-clean filter column (8 ml) for 0.5 h. Glacial acetic acid (139 mg) in dichloromethane (1 ml) was added followed by MP-Cyanoborohydride (Argonaut Technologies) (330 mg) and the mixture shaken overnight. The mixture was filtered and the MP-Cyanoborohydride washed with methanol (2×4 ml). The filtrate was applied to a methanol conditioned SCX column (2 g) and eluted with methanol. The column was then eluted with 2% aqueous 0.880 ammonia in methanol and the filtrate evaporated under reduced pressure. The residue was purified by MDAP using the procedures detailed at the beginning of the experimental to afford (3-chlorophenyl)-5-{[(2-fluoropyridin-4-ylmethyl)-amino]-methyl}-4-trifluoromethyl-pyrimidin-2yl}-amine, formate (50 mg)

NMR (MeOD) δ 3.88 (2H, s), 3.95 (2H, s), 6.99 (1H, d), 7.13 (1H, s), 7.26 (1H, t), 7.33 (1H, d), 7.55 (1H, d), 7.96 (1H, s), 8.13 (2H, d), 8.79 (1H, s).

LC/MS, t=3.17 min, molecular ion observed [MH⁺] 412.

### EXAMPLE 97

### (4-tert-Butyl-5-morpholin-4-ylmethyl-pyrimidin-2-yl)-(3-chloro-phenyl)-amine hydrochloride

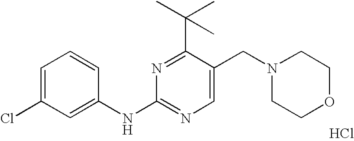

A mixture of (4-tert-butyl-2-(3-chloro-phenylamino)-pyrimidine-5-carbaldehyde (100 mg), morpholine (0.120 ml), and glacial acetic acid (0.0198 ml) in THF (2 ml) was stirred with 4A molecular sieves (65 mg) at 21° C. for 2 hours. Sodium triacetoxyborohydride (103 mg) and THF (2 ml) was added and stirring continued for 16 hours. The reaction mixture was filtered through a bed of Kieselguhr and this washed with EtOAc (10 ml). The solution was then washed with aqueous 1M NaOH and backwashed with EtOAc. The combined organics were washed with brine, dried (MgSO₄), and the volatiles were removed in vacuo to yield the crude product. The crude product was purified using MDAP. Excess ethereal HCl was added to the product and the resultant precipitate was filtered off, dried at 50° C. under vacuum to yield the title compound as a white solid (12 mg).

NMR (d⁶-DMSO) δ 2.50 (9H, s), 3.25-3.43 (4H, m), 3.80-3.98 (4H, m), 4.58 (2H, brs), 7.00 (1H, d), 7.31 (1H, t), 7.60 (1H, m), 8.14 (1H, s), 8.88 (1H, s), 10.00 (1H, s), 10.20 (1H, s).

LC/MS t=3.93 min [MH⁺]=361 consistent with molecular formula C₁₉H₂₅³⁵ClN₄O.HCl

The products in Table 4 were prepared in a similar method to the preparation of Example 97 from 2-(3-chloro-phenylamino)-4-isopropyl-pyrimidine-5-carbaldehyde or 4-tert-butyl-2-(3-chloro-phenylamino)-pyrimidine-5-carbaldehyde as appropriate.

TABLE 4

| | | | LC/MS |
|---|---|---|---|
| | | | Retention Time, |
| Ex- | | | t (min) [MH⁺] |
| am- | | | Molecular formula |
| ple | | | of free base |
| No | Structure | Name | NMR data |
| 98 | 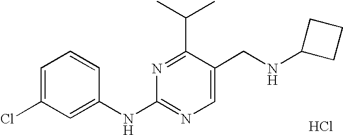 | (3-Chloro-phenyl)-{5-[(cyclobutylamino)-methyl]-4-isopropyl-pyrimidin-2-yl}-amine hydrochloride | NMR (d⁶-DMSO) δ1.23 (6 H, d), 1.48-1.74 (4 H, m),2.04-2.15 (2 H, m),3.11-3.20 (1 H, m), 3.28-3.40(2 H, m), 3.53 (H, s),6.93 (1 H, d), 7.28(1 H, t), 7.64 (1 H,d), 8.17 (1 H, s), 8.29(1 H, s), 9.69 (1 H, s). |
| 99 | 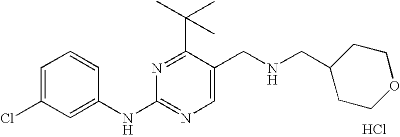 | (4-tert-Butyl-5-{[(tetrahydro-pyran-4-ylmethyl)-amino]-methyl}-pyrimidin-2-yl)-(3-chloro-phenyl)-aminehydrochloride | LC/MS t = 2.76. [MH⁺] = 389C₂₁H₂₉³⁵ClN₄ONMR (MeOD) δ 1.33-1.46(4 H, m),1.48 (9 H, s), 1.76(2 H, d),2.00-2.10 (1 H, m), 3.40-3.50(2 H, m), 3.98 (2 H, dd),4.46 (2 H, s), 7.00 (1 H, d),7.26 (1 H, t), 7.48 (1 H, d),8.06 (1 H, t), 8.49 (1 H, s). |
| 100 | 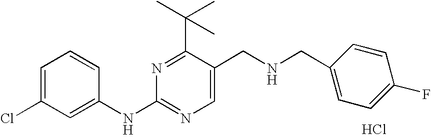 | (4-tert-Butyl-5-[(4-fluoro-benzylamino)-methyl]-pyrimidin-2-yl}-(3-chloro-phenyl)-amine hydrochloride | LC/MS t = 3.16. [MH⁺] = 399C₂₂H₂₄³⁵ClFN₄NMR (MeOD) δ 1.36 (9 H, s),4.40 (2 H, s) 4.43 (2 H, s), 6.99(1 H, d), 7.22-7.28 (3 H, m),7.46 (1 H, d),7.58-7.62 (2 H, m), 8.04 (1 H, t),8.44 (1 H, s). |
| 101 | 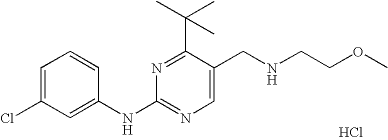 | (4-tert-Butyl-5-[(2-methoxy-ethylamino)-methyl]-pyrimidin-2-yl}-(3-chloro-phenyl)-aminehydrochloride | LC/MS t = 2.70 [MH⁺] = 409C₁₈H₂₅³⁵ClN₄ONMR (DMSO) δ 1.42 (9 H, s),3.14-3.34 (7 H, m), 3.62 (2 H, s),6.98 (1 H, d), 7.30 (1 H, t),7.60 (1 H, d),8.14 (1 H, s), 8.57 (1 H, s),9.90 (1 H, brs). |
| 102 | 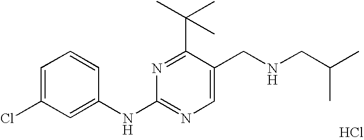 | (4-tert-Butyl-5-(isobutylamino-methyl)-pyrimidin-2-yl]-(3-chloro-phenyl)-aminehydrochloride | LC/MS t = 2.88 [MH⁺] = 347.C₁H₂₇³⁵ClN₄NMR (DMSO) δ 0.89 (6 H, d),1.45 (9 H, s), 1.73(1 H, quintet), 2.47 (2 H,3.86 (2 H, s), 6.95 (1 H, d),7.28 (1 H, t), 7.60 (1 H, d),8.13 (1 H, s), 8.48 (1 H, s),9.67 (1 H, s). |
| 103 | 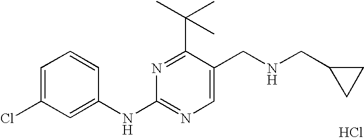 | (4-tert-Butyl-5-[(cyclopropylmethyl-amino)-methyl]-pyrimidin-2-yl}-(3-chloro-phenyl)-aminehydrocholoride | LC/MS t = 2.81 [MH⁺] = 345.C₁₉H₂₅³⁵ClN₄NMR (DMSO) δ 0.40-0.45 (2 H, m),0.59-0.64 (2 H, m),1.18 (1 H, t), 1.42 (9 H, s),2.99 (2 H, q), 4.34 (2 H, t),7.00 (1 H, d),7.31 (1 H, t), 7.61(1 H, d), 8.13 (1 H, s), 8.72 (1 H, s), 9.22 (2 H, brs),9.91 (1 H, s). |
| 104 | 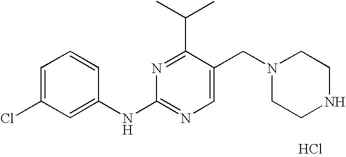 | (3-Chloro-phenyl)-(4-isopropyl-5-piperazin-1-ylmethyl-pyrimidin-2-yl)-aminehydrochloride | C₁₈H₂₄³⁵ClN₅NMR (d⁶-DMSO) δ 1.25 (6 H, d),2.60-2.92 (3 H, m), 3.17-3.82(8 H, m), 7.00 (1 H, d),7.31 (1 H, t), 7.64 (1 H, t),8.13 (1 H, s), 8.86 (1 H, brs),10.00 (1 H, s), 11.70 (1 H, brs). |

### EXAMPLE 105

### (5-Aminomethyl-4-trifluoromethyl-pyrimidin-2-yl)-(3-chloro-phenyl)-amine

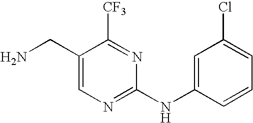

A solution of (5-bromomethyl-4-trifluoromethyl-pyrimidin-2-yl)-(3-chloro-phenyl)-amine, (Intermediate 13) (73.2 mg) in tetrahydrofuran (3 ml) was added dropwise to a stirred mixture of 880 ammonia (2 ml) and tetrahydrofuran (5 ml). The mixture was stirred at room temperature for 2 hrs, evaporated to dryness and re-evaporated from a mixture of dichloromethane and triethylamine. The mixture was taken up in dichloromethane containing a small amount of methanol and chromatographed on silica gel eluting with dichloromethane/methanol 5:1 to give the title compound as a pale yellow solid (31 mg).

NMR (DMSO-d6) F1577 δ 2.83 (2H, br s), 3.82 (2H, s), 7.04 (1H, dd), 7.34 (1H, t), 7.66 (1H, dd), 7.99 (1H, m), 8.92 (1H, s), 10.3 (1H, s).

LC/MS CF100425-1, t=2.45 min, molecular ion observed [M−H]− 301, consistent with C₁₂H₁₀³⁵ClF₃N₄.

Formulations for pharmaceutical use incorporating compounds of the present invention can be prepared in various forms and with numerous excipients. Examples of such formulations are given below.

### EXAMPLE 106

### Inhalant Formulation

A compound of formula (I) or a pharmaceutically acceptable derivative thereof, (1 mg to 100 mg) is aerosolized from a metered dose inhaler to deliver the desired amount of drug per use.

### EXAMPLE 107

### Tablet Formulation

| | |
|---|---|
| Tablets/Ingredients | Per Tablet |

| | | | |
|---|---|---|---|
| 1. | Active ingredient | 40 | mg |
| | (Compound of formula (I) or pharmaceutically | | |
| | acceptable derivative) | | |
| 2. | Corn Starch | 20 | mg |
| 3. | Alginic acid | 20 | mg |
| 4. | Sodium Alginate | 20 | mg |
| 5. | Mg stearate | 1.3 | mg |

### Procedure for Tablet Formulation:

Ingredients 1, 2, 3 and 4 are blended in a suitable mixer/blender. Sufficient water is added portion-wise to the blend with careful mixing after each addition until the mass is of a consistency to permit its conversion to wet granules. The wet mass is converted to granules by passing it through an oscillating granulator using a No. 8 mesh (2.38 mm) screen. The wet granules are then dried in an oven at 140° F. (60° C.) until dry. The dry granules are lubricated with ingredient No. 5, and the lubricated granules are compressed on a suitable tablet press.

### EXAMPLE 108

### Parenteral Formulation

A pharmaceutical composition for parenteral administration is prepared by dissolving an appropriate amount of a compound of formula (I) in polyethylene glycol with heating. This solution is then diluted with water for injections Ph Eur. (to 100 ml). The solution is then rendered sterile by filtration through a 0.22 micron membrane filter and sealed in sterile containers.

It is to be understood that the present invention covers all combinations of particular and preferred groups described herein above.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I); 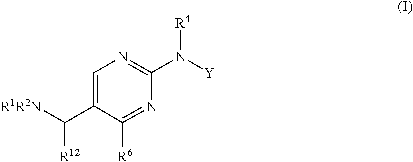
wherein:
Y is phenyl, unsubstituted or substituted with one, two or three substituents;
R¹ is selected from hydrogen, C₁₋₆ alkyl, C₃₋₆ cycloalkyl, or halosubstituted C₁₋₆ alkyl;
R² is (CH₂)ₘR³ where m is 0 or 1;
or R¹ and R² together with N to which they are attached form an unsubstituted or substituted 4- to 8-membered non-aromatic heterocyclyl ring;
R³ is hydrogen, an unsubstituted or substituted 4- to 8-membered non-aromatic heterocyclyl group, an unsubstituted or substituted C₃₋₈ cycloalkyl group, an unsubstituted or substituted straight or branched C₁₋₁₀ alkyl, an unsubstituted or substituted C₅₋₇ cycloalkenyl, R⁵; or R³ is an unsubstituted or substituted 5- to 6-membered aromatic heterocyclyl group, or group A: 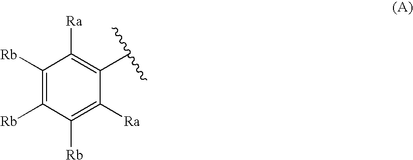
R⁴ is selected from hydrogen, C₁₋₆ alkyl, C₃₋₆ cycloalkyl, or halosubstitutedC₁₋₆ alkyl, COCH₃, or SO₂Me;
R⁵ is 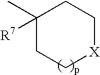
wherein p is 0, 1 or 2, and X is CH₂, O, S, SO or SO₂;
R⁸ is halo, an substituted or unsubstituted (C₁₋₆)alkyl, (C₃₋₆)cycloalkyl, 4- to 7-membered non aromatic heterocyclyl group;
R⁷ is OH, C₁₋₆alkoxy, NR^{8a}R^{8b}, NHCOR₉, NHSO₂R⁹, SOqR⁹;
R^{8a} is H or C₁₋₆alkyl;
R^{8b} is H or C₁₋₆alkyl;
R⁹ is C₁₋₆alkyl;
Ra is independently selected from hydrogen, fluoro, chloro or trifluoromethyl;
Rb is independently selected from hydrogen, C₁₋₆ alkyl, C₁₋₆ alkoxy, haloC₁₋₆ alkoxy, hydroxy, cyano, halo, sulfonyl, CONH₂, COOH or NHCOOC₁₋₆alkyl;
R¹² is hydrogen or C₁₋₆alkyl;
q is 0, 1 or 2;
or a pharmaceutically acceptable derivative thereof,
wherein the compound is not (5-{([bis-(2-methoxy-ethyl)-amino]-methyl}-4-trifluoromethyl-pyrimidin-2-yl)-(3-chlorophenyl)-amine or (1-[2-(3-chloro-phenylamino)-4-trifluoromethyl-pyrimidin-5-ylmethyl]-piperidin-4-yl-methanol, formate.

2. A compound as claimed in claim 1 wherein the compound of formula (I) is a compound of formula (Ia): 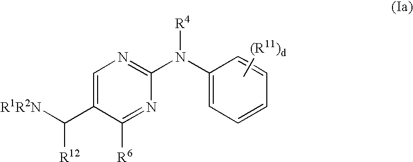
wherein;
R¹ is selected from hydrogen, C₁₋₆ alkyl, C₃₋₆ cycloalkyl and halosubstitutedC₁₋₆ alkyl;
R² is (CH₂)ₘR³ where m is 0 or 1;
or R¹ and R² together with N to which they are attached form a 4- to 8-membered non-aromatic ring selected from azetidinyl, pyrrolidinyl, morpholinyl, piperizinyl, piperidinyl, thiomorpholinyl, tetrahydropyridinyl, azapine, oxapine, azacyclooctanyl, azaoxacyclooctanyl and azathiacyclooctanyl any of which can be unsubstituted or substituted by one, two or three substituents selected from C₁₋₆ alkyl, C₁₋₆ alkylOH, C₁₋₆ alkoxy, a hydroxy group, a cyano group, halo, sulfonyl group, methylsulfonyl, NR^{8a}R^{8b}, NHCOCH₃, (═O), —CONHCH₃ and NHSO₂CH₃, C(O)OC₁₋₆alkyl;
R³ is hydrogen, 2- or 3-azetidinyl, oxetanyl, thioxetanyl, thioxetanyl-s-oxide, thioxetanyl-s,s-dioxide, dioxalanyl, pyrrolidinyl, tetrahydrofuranyl, tetrahydrothiophenyl, tetrahydrothiophenyl-s-oxide, tetrahydrothiophenyl-s,s-dioxide, morpholinyl, piperidinyl, piperazinyl, tetrahydropyranyl, tetrahydrothiopyranyl, tetrahydrothiopyranyl-s-dioxide, tetrahydrothiopyranyl-s,s-dioxide, thiomorpholinyl, thiomorpholinyl-s,s-dioxide, tetrahydropyridinyl, dioxanyl, tetrahydrothiopyran 1,1 dioxide, azapine, oxapine, azacyclooctanyl, azaoxacyclooctanyl, azathiacyclooctanyl, oxacylcooctanyl, thiacyclooctanyl, a C₃₋₈ cycloalkyl group, a straight or branched C₁₋₁₀ alkyl, a C₅₋₇ cycloalkenyl or R⁵, any of which can be unsubstituted or substituted by one, two or three substituents selected from C₁₋₆ alkyl, C₁₋₆ alkoxy, a hydroxy group, a cyano group, halo, sulfonyl group, methylsulfonyl, NR^{8a}R^{8b}, NHCOCH₃, (═O), and —CONHCH₃ and when R³ is alkyl it can be phenyl or phenyl substituted by halo, hydroxy or cyano;
or R³ is group A or selected from furanyl, dioxalanyl, pyrrolyl, oxazolyl, thiazolyl, imidazolyl, oxadiazolyl, thiadiazolyl, triazolyl, triazinyl, isothiazolyl, isoxazolyl, thienyl, pyrazolyl, tetrazolyl, pyridinyl, pyrizinyl, pyrimidinyl, pyrazinyl, triazinyl, or tetrazinyl any of which can be unsubstituted or substituted by one, two or three substituents selected from C₁₋₆ alkyl, C₁₋₆ alkoxy, a hydroxy group, a cyano group, halo, sulfonyl group, methylsulfonyl, NR^{8a}R^{8b}, NHCOCH₃, (═O), and —CONHCH₃; 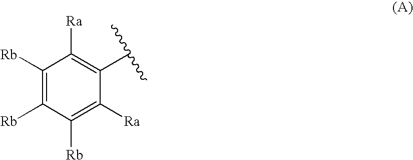
R¹¹ is selected from C₁₋₆ alkyl, halosubstitutedC₁₋₆ alkyl, C₁₋₆ alkoxy, a hydroxy group, a cyano group, halo, a C₁₋₆alkyl sulfonyl group, —CONH₂₁—NHCOC₁₋₆alkyl, —COOH, —CH₂COOH, halosubstitutedC₁₋₆ alkoxy, SC₁₋₆alkyl and SO₂NR^{8a}R^{8b};
R⁴ is selected from hydrogen, C₁₋₆ alkyl, C₃₋₆ cycloalkyl, or halosubstitutedC₁₋₆ alkyl, COCH₃, and SO₂Me;
R⁵ is 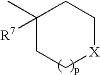
wherein p is 0, 1 or 2 and X is CH₂, O, S, SO or SO₂;
R⁶ is halo, a substituted or unsubstituted (C₁₋₆)alkyl, (C₃₋₆)cycloalkyl, 4- to 7-membered non aromatic heterocyclyl group;
R⁷ is OH, C₁₋₆alkoxy, NR^{8a}R^{8b}, NHCOR⁹, NHSO₂R⁹, SOqR⁹;
R^{8a} is H or C₁₋₆alkyl;
R^{8b} is H or C₁₋₆alkyl;
R⁹ is C₁₋₆alkyl;
R¹² is hydrogen or C₁₋₆alkyl;
Ra is independently selected from hydrogen, fluoro, chloro or trifluoromethyl;
Rb is independently selected from hydrogen, C₁₋₆ alkyl. C₁₋₆ alkoxy, haloC₁₋₆ alkoxy, hydroxy, cyano, halo, sulfonyl, CONH₂, COOH or NHCOOC₁₋₆alkyl;
q is 0, 1 or 2;
d is 0, 1, 2 or 3
or a pharmaceutically acceptable derivative thereof.
wherein the compound is not
(5-{[bis-(2-methoxy-ethyl)-amino]-methyl}-4-trifluoromethyl-pyrimidin-2-yl)-(3-chlorophenyl)-amine or {1-[2-(3-chloro-phenylamino)-4-trifluoromethyl-pyrimidin-5-ylmethyl]-piperidin-4-yl}-methanol, formate.

3. A compound as claimed in claim 1 wherein the compound of formula (I) is a compound of formula (Ib): 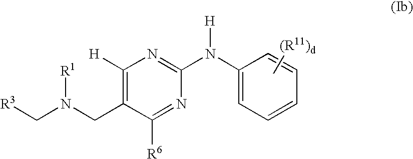
wherein;
R¹ is hydrogen or methyl;
R³ is an unsubstituted or substituted 4- to 8-membered non-aromatic heterocyclyl group an unsubstituted or substituted C₃₋₈ cycloalkyl group, an unsubstituted or substituted straight or branched C₁₋₁₀ alkyl;
R⁶ is an substituted or unsubstituted (C₁₋₆)alkyl, (C₃₋₆)cycloalkyl, or 4- to 7-membered non aromatic heterocyclyl group;
R¹¹ is selected from halo cyano, methyl, trifluoromethyl, methoxy, trifluoromethoxy or SCH₃;
d is 0, 1, 2 or 3;
or a pharmaceutically acceptable derivative thereof wherein the compound is not
{1-[2-(3-chloro-phenylamino)-4-trifluoromethyl-pyrimidin-5-ylmethyl]-piperidin-4-yl}-methanol, formate.

4. A compound as claimed in claim 1 wherein the compound of formula (I) is a compound of formula (Ic): 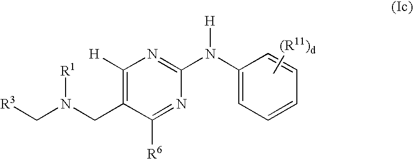
wherein
R¹ is hydrogen or methyl.
R³ is group A, pyridinyl, or pyrimidinyl, any of which can be optionally substituted; 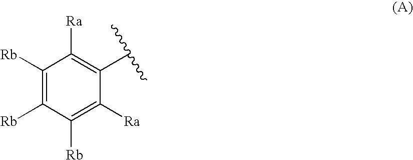
Ra is independently selected from hydrogen, fluoro, chloro or trifluoromethyl;
Rb is independently selected from hydrogen, C₁₋₆ alkyl, C₁₋₆ alkoxy, haloC₁₋₆ alkoxy, hydroxy, cyano, halo, sulfonyl, CONH₂, COOH or NHCOOC₁₋₆alkyl;
R⁶ is an substituted or unsubstituted (C₁₋₆)alkyl, (C₃₋₆)cycloalkyl or 4- to 7-membered non aromatic heterocyclyl group;
R¹¹ is selected from halo, cyano, methyl, trifluoromethyl, methoxy, trifluoromethoxy SCH₃;
d is 0, 1, 2 or 3;
or a pharmaceutically acceptable derivative thereof.

5. A compound as claimed in claim 1 wherein R⁶ is either cyclopropyl, isopropyl, tert-butyl or trifluoromethyl.

6. (canceled)

7. A pharmaceutical composition comprising a compound as claimed in claim 1 or a pharmaceutically acceptable derivative thereof and a pharmaceutical carrier or diluent thereof.

8. A pharmaceutical composition as claimed in claim 7 further comprising a second therapeutic agent.

9. A method of treating a mammal suffering from a condition which is mediated by the activity of cannabinoid 2 receptors which comprises administering to said subject a therapeutically effective amount of a compound of formula (I) as claimed in claim 1 or a pharmaceutically acceptable derivative thereof.

10. A compound of formula (I) as claimed in claim 1 or a pharmaceutically acceptable derivative thereof for use as a medicament in the treatment of pain.

11. A pharmaceutical composition comprising a compound as claimed in claim 1 or a pharmaceutical derivative thereof and at least one Cox-2 inhibitor.

12. A pharmaceutical composition comprising a compound as claimed in claim 1 or a pharmaceutical derivative thereof and at least one PDE4 inhibitor.

13. The method of claim 9 wherein the condition is selected from an immune disorder, an inflammatory disorder, pain, rheumatoid arthritis, multiple sclerosis, osteoarthritis or osteoporosis.
